(19) United States
(12) Patent Application Publication
(10) Pub. No.: US 2006/0217345 A1
(43) Pub. Date: Sep. 28, 2006

(54) Beta-L-nucleosides and use thereof as pharmaceutical agents for the treatment of viral diseases

(76) Inventors: Eckart Matthes, Eggersdorf (DE); Martin Janta-Lipinski, Berlin (DE); Hans Will, Hamburg (DE); Huseyin Sirma, Elmshorn (DE); Lin Li, Hamburg (DE)

Correspondence Address: NILS H. LJUNGMAN;NILS H. LJUNGMAN & ASSOCIATES, P.O. BOX 130, GREENSBURG, PA, 15601-0130, US

(21) Appl. No.: 11/373,062

(22) Filed: Mar. 10, 2006

Related U.S. Application Data
(63) Continuation-in-part of application No. PCT/DE04/02051, filed on Sep. 13, 2004.

(30) Foreign Application Priority Data
Sep. 12, 2003 (DE) 103 42 510.1
Sep. 12, 2003 (DE) 103 42 509.8

(51) Int. Cl. A61K 31/7072; A61K 31/675; A61K 31/513; C07H 19/04; C07H 19/12; C07F 9/6512

(52) U.S. Cl. 514/49; 514/269; 536/28.3; 544/243; 536/26.1; 544/310

(57) ABSTRACT

Nucleoside analogs, nucleic acids and pharmaceutical agents comprising same, and to the use of said nucleoside analogs, nucleic acids and pharmaceutical agents in the diagnosis, prophylaxis or therapy of a viral, bacterial, fungicidal and/or parasitic infection, or of cancer, particularly of hepatitis infections. The invention also relates to a method for the preparation of said nucleoside analogs and to a kit and the use thereof in the prophylaxis and therapy of viral diseases, particularly of hepatitis infections. As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

### CONTINUING APPLICATION DATA

The present application is a Continuation-In-Part application of International Patent Application No. PCT/DE2004/002051, filed on Sep. 13, 2004, which claims priority from Federal Republic of Germany Patent Applications No. 103 42 510.1 and No. 103 42 509.8, each filed on Sep. 12, 2003. International Patent Application No. PCT/DE2004/002051 was pending as of the filing date of this application. The United States was an elected state in International Patent Application No. PCT/DE2004/002051.

### DESCRIPTION

The application relates to new β-L-5-methylcytosine nucleosides having a structure as apparent from the following formulas I and II,

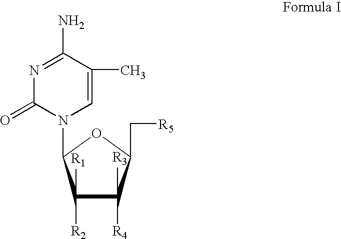

wherein

R₁═H, OH, F

R₂═H, OH

R₃═H

R₄═H, OH, N₃

and R₅═OH, O-acetyl, O-palmitoyl, alkoxycarbonyl, carbamate, phosphonate, monophosphate, diphosphate or triphosphate, and

if R₁, R₂ and R₃═H, then R₄═H, OH or N₃, and

if R₁═OH or F, then R₂ and R₃═H and R₄═OH,

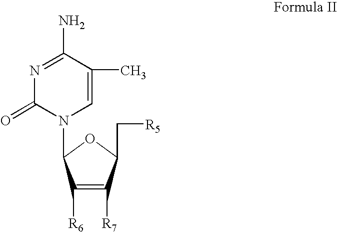

wherein

R₆═H, F, and R₇═H,

and their use as pharmaceutical active substances or agents in the prophylaxis and/or treatment of infections caused in particular by hepatitis B virus (HBV).

The β-L-5-methylcytosine nucleosides and the acceptable salts or prodrugs thereof can be used alone or in combination with other β-L-nucleosides, with 3-deazauridine or with other anti-HBV-effective compounds.

Fields of use of the invention are medicine and the pharmaceutical industry.

### BACKGROUND INFORMATION

HBV is the agent that triggers hepatitis B—an infectious disease, the chronic course of which affects about 350 million people worldwide, and particularly in Southeast Asia, Africa and South America. In a large number of cases, hepatitis B virus infections lead to eventual death as a result of liver function failure. Moreover, the chronic course is associated with a massively increased risk of primary liver carcinoma which, in China alone, results in about one million new cases of disease each year.

While the precise mechanism through which HBV can induce liver tumors remains unknown, it must be assumed that tumor induction is closely associated with HBV-induced chronic inflammation, developing cirrhosis and regeneration processes of the liver tissue.

The vaccine produced by genetic engineering, which has been available for many years, is not suitable for the treatment of hepatitis B because it fails to help persons already infected and is unable to stop the chronic course mentioned above.

In recent years, α-interferon produced by genetic engineering, in particular, has been found useful in the treatment of HBV infections. It is a cytokine with broad antiviral and immunomodulating activity. However, it is effective in only about 33% of the patients, entails considerable side effects, and cannot be administered on the oral route.

One nucleoside derivative applied with success and approved by the US Food and Drug Administration, as well as in Germany, is lamivudine (β-L-2′,3′-dideoxy-3′-thiacytidine), also known as thiacytidine (3TC), which has been described by Liotta et al. in U.S. Pat. No. 5,539,116. It is remarkable for its high efficacy both in HbeAg-positive and HbeAg-negative patients and has scarcely any side effects.

Although rapid decline of HBV DNA and normalization of the alanine transferase activity in serum is found in such treatment, HBV apparently cannot be completely eliminated from the liver under such therapy, so that re-onset of a hepatitis B infection is possible in many cases even after completion of a one-year treatment. Attempts are being made to prevent the above course by extending the treatment to several years, in the hope that HBV could be eliminated completely (Alberti et al., J Med Virol 2002, 67: 458-462).

However, such therapies are associated with an increasing risk of resistance to lamivudine, which can be as high as 45-55% after the second year of treatment (Liaw et al., Gastroenterology 2000, 119: 172-180).

The development of additional effective compounds is therefore an urgent necessity in order to replace the monotherapy by a combination therapy which not only can be more effective but can also substantially reduce the risk of resistance, as has been found in long-term treatment of HIV infections (Richman, Nature 2001, 410: 995-1000).

Lamivudine belongs to a group of so-called β-L-nucleosides. They are enantiomeric compounds of naturally occurring β-D-nucleosides and, for a long time, have been regarded as defying enzymatic metabolization and therefore as inactive in biological systems.

This dogma was relativized for the first time in 1992 by the findings of Spadari et al. who had discovered that β-L-thymidine, while not being reacted by cellular thymidine kinase 1, is a substrate of the corresponding enzyme of herpes simplex virus 1 (Spadari et al., J Med Chem 1992, 35: 4214-4220). It has later been found that β-L-nucleosides can be substrates or inhibitors not only to some viral but also to some cellular enzymes (Review: Maury, Antiviral Chem Chemother 2000, 11: 165-190).

In the following years, a variety of β-L-nucleoside analogs have been synthesized in pure form, among which—in addition to the above-mentioned lamivudine (3TC; β-L-2′,3′-dideoxy-3′-thiacytidine; Jeong et al., J Med Chem 1993, 36: 181-195)-emtricitabine (L-FTC; β-L-2′,3′-dideoxy-5-fluoro-3′-thiacytidine; Furman et al., Antimicrob Agents & Chemother 1992, 36: 2686-2692), β-L-2′-fluoro-5-methylarabinofuranosyluracil (L-FMAU; clevudine; Chu et al., Anti-microb Agents & Chemother 1995, 39: 979-981), β-L-2′,3′-dideoxycytidine and β-L-2′,3′-dideoxy-5-fluorocytidine (L-ddC, L-FddC; Lin et al., J Med Chem 1994, 37: 798-803), β-L-2′,3′-dideoxy-2′,3′-didehydrocytidine (L-ddeC; Lin et al., J Med Chem 1996, 39: 1757-1759), and β-L-thymidine (L-TdR; telbivudine; by Janta Lipinski et al., J Med Chem. 1998, 41: 2040-2046; Bryant et al., Antimicrob Agents & Chemother 2001, 45: 229-235) have been found to be the most effective and promising inhibitors of HBV replication in vitro and in vivo, which are remarkable for their—in some cases—extremely low cytotoxicity. Among the D-nucleosides, entecavir (BMS 200475), a carbocyclic deoxyguanosine derivative (Innaimo et al., Antimicrob Agents & Chemother 1997, 41: 1444-1448), should be mentioned in particular, which has proven to be superior to lamivudine in the treatment of hepatitis B in an initial clinical study (Lai et al., Gastroenterology 2002, 123: 1831-1838).

Further syntheses of L-nucleosides have been described in Mugnaini et al., Bioorg Med Chem 2003, 11: 357-366; Marquez et al., J Med Chem 1990, 33: 978; Lee et al., Nucleosides & Nucleotides 1999, 18: 537-540; Faraj et al., Nucleosides & Nucleotides 1997, 16: 1287-1290; Song et al., J Med Chem 2001, 44: 3985-3993; Kotra et al., J Med Chem 1997, 40: 1944; Choi et al., Organic Lett 2002, 4: 305-307; Gumina et al., Curr Top Med Chem 2002, 2: 1065-1086; Holy, Tetrahedron Lett. 1971, 189-193; Holy, Collect Czech Chem Commun 1972, 37: 4072-4082; and, in addition, the following patents describe β-L-nucleosides as potential virustatic agents: Gosselin et al., U.S. Pat. No. 6,395,716, Schinazi et al., US 2002-0107221 A1; Chu et al., U.S. Pat. No. 5,565,438, U.S. Pat. No. 5,567,688, U.S. Pat. No. 5,587,362, WO 92/18517 of the Yale University and University of Georgia Research Foundation, Inc.

In addition to β-L-cytosine nucleosides with non-modified cytosine as in β-L-deoxycytidine (Bryant et al., Antimicrob Agents & Chemother 2001, 45: 229-235), β-L-2′,3′-dideoxycytidine (Lin et al., J Med Chem 1994, 37: 798-803), β-L-2′,3′-dideoxy-2′,3′-didehydrocytidine (Lin et al., J Med Chem 1996, 39: 1757-1759), β-L-2′-fluoroarabinofuranosylcytosine (FAC; Ma et al., J Med Chem 1996, 39: 2835-2843), β-L-arabinofuranosylcytosine (L-AraC; Chu et al., U.S. Pat. No. 5,567,688), β-L-2′,3′-dideoxy-2′,3′-didehydro-2′-fluorocytidine (L-2′FddeC; Lee et al., J Med Chem 1999, 42: 1320-1328), some 5-modified cytosine derivatives have also been synthesized and investigated, especially 5-fluorocytosine derivatives which are either more effective than compounds with non-modified bases, such as β-L-2′,3′-dideoxy-2′,3′-didehydro-5-fluorocytidine (L-FddeC; Lin et al., J Med Chem 1996, 39: 1757-1759), equally effective, such as β-L-2′,3′-dideoxy-5-fluorocytidine (L-FddC; Lin et al., J Med Chem 1994, 37: 798-803) or β-L-2′,3′-dideoxy-2′,3′-didehydro-2′-fluoro-5-fluorocytidine (L-2′F-ddeFC; Lee et al., J Med Chem 1999, 42: 1320-1328), less effective, such as β-L-deoxy-5-fluorocytidine (FdC; Bryant et al., Antimicrob Agents & Chemother 2001, 45: 229-235), or exhibit no effect with respect to HBV replication, such as β-L-2′-fluoroarabinofuranosyl-5-fluorocytosine (L-FAFC; Ma et al., J Med Chem 1996, 39: 2835-2843) or β-L-arabinofuranosyl-5-fluorocytosine (L-AraFC; Griffon et al., Eur J Med Chem 2001, 36: 447-460).

Likewise, the following 5-chloro-, bromo- and methyl-modified L-cytosine nucleosides have been described as ineffective or sparingly effective: β-L-deoxy-5-chlorocytidine (CldC; Bryant et al., Antimicrob Agents & Chemother 2001, 45: 229-235), β-L-2′-fluoroarabinofuranosyl-5-chlorocytidine, β-L-2′-fluoroarabinofuranosyl-5-bromocytosine (L-FAC1C, L-FABrC; Ma et al., J Med Chem 1996, 39: 2835-2843), β-L-2′,3′-dideoxy-3′-thia-5-methylcytidine, β-L-2′,3′-dideoxy-3′-thia-5-bromocytidine, β-L-2′,3′-dideoxy-3′-thia-5-chlorocytidine and β-L-2′,3′-dideoxy-3′-fluoro-5-methylcytidine (Dong et al., Proc Natl Acad Sci USA 1991, 88: 8495-8499; Matthes et al., unpublished) so that, indeed, 5-fluoro modifications of β-L-cytosine nucleosides are known at present, but only a few other 5-modifications have been prepared, and testing thereof has not furnished significant effects on HBV replication.

Some of the above-mentioned L-nucleosides are not only effective inhibitors of HBV replication, but also of HIV replication. Thus, for example, lamivudine has also been approved for the treatment of HIV infections. Other β-L-cytosine nucleosides already mentioned above, such as L-ddC, L-5FddC, L-FddeC, FTC, are also strong inhibitors of HIV replication, whose importance for therapy is to have new effective compounds available for combination therapy, thus providing the capability of coping with development of resistance (Menendez-Arias, Trends Pharmacol Sci 2002, 23: 381-388).

In addition, there are a number of β-L-nucleosides inhibiting HBV replication only (e.g. L-FMAU, L-TdR, L-CdR, L-3′FddC, L-d4C) and others inhibiting HIV replication only (e.g. abacavir).

All of the above-mentioned β-L-nucleosides are incorporated by HBV- or HIV-infected cells and must be converted into the nucleoside triphosphates by cellular enzymes. It is only in this form where the nucleosides can bind their actual target, i.e. the HBV DNA polymerase or reverse transcriptase, in competition with normal substrates and give strong inhibition. As a consequence, the viral genomes can no longer by synthesized, and virus production comes to a standstill. Such inhibition must be selective, i.e., must be restricted to the viral polymerases and must not co-involve the cellular DNA polymerases, because otherwise—as a consequence of inhibition of the synthesis of cellular DNA—growth of rapidly proliferating cells would be impaired.

At least one embodiment is based on the object of developing new, antivirally effective β-L-methylcytosine nucleosides which, in particular, are effective against hepatitis B virus infections and HIV infections and exhibit high efficacy against said infections, while having good tolerability and low toxicity.

Surprisingly, β-L-5-methylcytosine nucleosides in accordance with formula I or formula II,

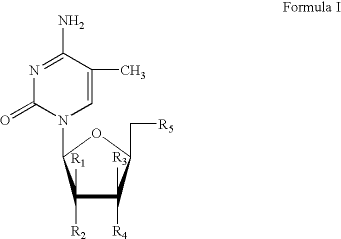

wherein

R₁═H, OH, F

R₂═H, OH

R₃═H

R₄═H, OH, N₃

and R₁═OH, O-acetyl, O-palmitoyl, alkoxycarbonyl, carbamate, phosphonate, monophosphate, diphosphate or triphosphate, and

if R₁, R₂ and R₃═H, then R₄═H, OH or N₃, and

if R₁═OH or F, then R₂ and R₃═H and R₄═OH,

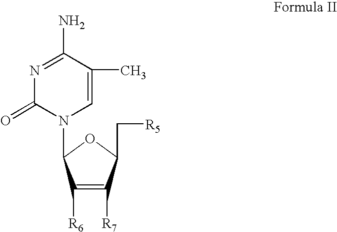

wherein

R₆═H, F, and R₇═H,

exhibit high antiviral activity especially against HBV and HIV.

The following are particularly effective:

β-L-5-methyldeoxycytidine (β-L-MetCdR),

β-L-2′,3′-dideoxy-5-methylcytidine (β-L-ddMetC),

β-L-2′,3′-didehydro-2′,3′-dideoxy-5-methylcytidine (β-L-ddeMetC),

β-L-arabinofuranosyl-5-methylcytosine (β-L-AraMetC),

β-L-2′,3′-didehydro-2′,3′-dideoxy-2′-fluoro-5-methylcytidine (β-L-FddeMetC),

β-L-2′-fluoroarabinofuranosyl-5-methylcytosine (β-L-FMAC), and

β-L-3-azido-2′,3′-dideoxy-5-methylcytidine (L-N3MetCdR),

the efficacy of which can be strongly increased by simultaneous application of 3-deazauridine.

In contrast to the β-L-series, some 5-methylcytidine derivatives of the above-mentioned compounds in the β-D-series are known.

Here, the triphosphates of β-D-AraMetC, β-D-ddeMetC, β-D-FMetC, β-D-AzMetC have been found to be highly effective inhibitors of HBV DNA polymerase (Matthes et al., Antimicrob Agent & Chemother 1991, 35: 1254-1257), the nucleosides of which, however, have only moderate intracellular and in vivo efficacy (Matthes et al., Biochemical Pharmacol 1992, 43: 1571-1577), probably as a consequence of the high deamination rate caused by deoxycytidine deaminase, giving rise to the fact that it is almost exclusively thymidine analogs that are present in the cells.

In contrast, it has been demonstrated for the enantiomeric β-L-deoxycytidine and a variety of modified derivatives thereof (e.g. β-L-AraC, 3TC, β-L-FTC, β-L-FddC, β-L-FddeC) that such deamination can no longer proceed at the nucleoside stage (Review: Maury, Antiviral Chem Chemother 2000, 11: 165-190), so that the first and most difficult activation step of β-L-5-methylcytosine nucleosides to form the monophosphate is accomplished by the non-stereospecific deoxycytidine kinase rather than by thymidine kinase 1 which is barely capable of phosphorylating L-thymidine derivatives (Focher et al., Current Drug Targets—Infectious Disorders 2003, 3: 41-54).

3-Deazauridine activates the cellular deoxycytidine kinase and, in addition, the triphosphate thereof, formed intra-cellularly, is capable of inhibiting the cellular CTP synthase (Gao et al., Nucleosides Nucleotides Nucleic Acids 2000, 19: 371-377). As a consequence of the above two effects on the cellular deoxycytidine metabolism, 3-deazauridine gives rise to increased triphosphate levels of the β-L-5-methylcytosine nucleosides of at least one embodiment, thereby massively increasing their efficacy with respect to HBV replication.

Surprisingly, it was found that the nucleosides according to at least one embodiment, i.e., the β-L-methylcytosine nucleosides, can be used with high antiviral activity against selected viruses, especially against hepatitis viruses, preferably against hepatitis B virus. The nucleosides or nucleoside analogs of at least one embodiment are structures differing in some characteristics from naturally occurring nucleosides; however, analogy to naturally occurring nucleosides is given in at least two essential issues. On the one hand, a nucleobase is always required, optionally a modified one, which is needed as a binding site to the complementary viral DNA mother strand. On the other hand, a functional group in the former 5′ position must be present, allowing formation of a high-energy triphosphate from the nucleosides or nucleoside analogs of at least one embodiment. Regarding the inhibition of the viral enzyme DNA polymerase, use of the nucleoside triphosphate is possible only as late as in the chain extension process. However, direct administration of the active nucleoside triphosphate as active substance is less preferred because it undergoes degradation in the blood plasma by non-specific phosphatases to form the corresponding free nucleoside analogs. Moreover, due to their negative charge, triphosphates cannot permeate cell membranes, thus failing to reach the site of action inside the cell. Using their own viral thymidine kinase, some viruses, such as herpes viruses, are capable of metabolizing the nucleoside analogs of at least one embodiment in infected cells to form the respective nucleoside monophosphate which in turn is converted into the triphosphate, the actual effective metabolite, by the cellular enzyme.

The triphosphate as an alternative substrate competes with the natural substrate for incorporation in the DNA. In particular, the absence of a 3′-hydroxyl function or analogous group terminates any further chain extension. However, it is also possible that the triphosphate acts as a competitive inhibitor of viral DNA polymerase. Thus, impairment or complete termination of virus replication by the structure according to at least one embodiment is possible in many ways.

In a preferred embodiment of at least one embodiment, derivatives of the inventive nucleosides are used. This may concern structures having modifications which, in particular, increase the antiviral activity. However, this may also concern a salt, a phosphonate, a monophosphate, a diphosphate, a triphosphate, an ester or a salt of such ester. Advantageously, such compounds can be used effectively in antiviral prophylaxis and therapy and exhibit only minor or no side effects at all.

In a preferred embodiment of at least one embodiment the β-L-5-methylcytosine nucleosides are β-L-5-methyldeoxycytidine, 5-methylcytosine, β-L-2′,3′-dideoxy-5-methylcytidine (L-ddMetC), β-L-2′,3′-didehydro-2′,3′-dideoxy-5-methylcytidine (L-ddeMetC), β-L-arabinofuranosyl-5-methylcytosine (L-MetaraC), β-L-2′-fluoroarabinofuranosyl-5-methylcytosine (L-FMAC), β-L-2′,3′-didehydro-2′,3′-dideoxy-2′-fluoro-5-methylcytidine (L-FddeMetC) and/or β-L-3′-azido-2′,3′-dideoxy-5-methylcytidine (L-AzMetdC), wherein the C-5 OH group of the sugar components can be replaced by O-acetyl, O-palmitoyl, alkoxycarbonyloxy, phosphonate, mono-, di-, triphosphate or another protective group and re-converted into the hydroxyl group in a subsequent reaction, so that the above modifications represent so-called prodrugs of the β-L-5-methylcytosine nucleosides, which advantageously have a particularly high antiviral activity.

Particularly preferred are β-L-5-methyldeoxycytidine (L-MetCdR), β-L-2′,3′-dideoxy-5-methylcytidine (L-ddMetC), β-L-2′,3′-didehydro-2′,3′-dideoxy-5-methylcytidine (L-ddeMetC), β-L-arabinofuranosyl-5-methylcytosine (L-AraMetC) and β-L-2′,3′-didehydro-2′,3′-dideoxy-2′-fluoro-5-methylcytidine (L-FddeMetC), as well as β-L-2′-fluoroarabinofuranosyl-5-methylcytosine (L-FMAC) and β-L-3′-azido-2′,3′-dideoxy-5-methylcytidine (L-AzMetdC).

The preparation of the compounds according to at least one embodiment proceeds according to per se known procedures, using condensation of the sugar portion and the heterocycle or modification of the L-ribosyl residue.

The preferred compounds are particularly suitable for the treatment of clinical manifestation of viral hepatitis B infection in humans and for prophylactic treatment of hepatitis infections. The preferred compounds give particularly effective inhibition of the growth of DNA viruses at the stage of virus-specific transcription or translation. More specifically, the substances can influence viral growth via inhibition of the enzyme reverse transcriptase or via chain termination of a growing DNA chain. Furthermore, the structures of at least one embodiment can cause separation of a base pair and thus mispairing or shift of the arrangement in the growing DNA chain, or they prevent formation of an RNA-DNA hybrid, thus possibly giving rise to chain termination or inhibition or modification of viral replication. Provided the selected structures of at least one embodiment are not chain terminators, incorporation of a nucleoside of at least one embodiment in DNA by viral transcriptase may have an inhibiting effect because the nucleoside according to at least one embodiment introduces multiple mutations into the subsequent cycles of polymerization and accumulation, and some of such mutations result in inhibition of the virus. In a preferred fashion, however, the structures of at least one embodiment—provided they are not chain terminators or become incorporated in the DNA—produce an inhibiting effect by binding to the active or allosteric binding site of reverse transcriptase, thereby causing competitive or non-competitive inhibition. It goes without saying that the nucleosides of at least one embodiment have a very broad therapeutic spectrum. It is possible, for example, that the nucleosides in combination with other therapeutic, preferably antiviral, agents have a synergistic effect by increasing the therapeutic effect in an additive or non-additive fashion, particularly by increasing the therapeutic index and/or reducing the risk of toxicity inherent in each single compound. Accordingly, the nucleosides of at least one embodiment preferably can also be used in combination therapies, including a wide variety of combinations with well-known therapeutic agents and pharmaceutically acceptable carriers. Of course, veterinary uses are also possible, as well as feed additives for all vertebrates. Particularly preferred is the use in humans. According to the explications above, the nucleosides of at least one embodiment can be used as drugs in a particularly preferred fashion. To this end, the nucleosides can be used alone, as a salt or derivative or as a composition. Pharmaceutically tolerable salts of compounds of the present invention include those derived from pharmaceutically tolerable inorganic and organic acids and bases. Examples of suitable acids include hydrochloric, hydrobromic, sulfuric, nitric, perchloric, fumaric, maleic, phosphoric, glycolic, lactic, salicylic, succinic, p-toluenesulfonic, tartaric, acetic, citric, methanesulfonic, ethanesulfonic, formic, benzoic, malonic, naphthalene-2-sulfonic and benzenesulfonic acids. Preferred acids include hydrochloric, sulfuric, methanesulfonic and ethanesulfonic acids. Most preferred is methanesulfonic acid. Other acids, such as oxalic acid, although not being pharmaceutically tolerable themselves, can be used in the production of salts usable as intermediate products in obtaining the compounds of at least one embodiment and their pharmaceutically tolerable acid addition salts.

Salts derived from suitable bases include alkali metal (e.g. sodium), alkaline earth metal (e.g. magnesium), ammonium and N (C₁₋₄ alkyl)₄⁺ salts.

Combinations of substituents and variables presented by this invention are preferably those resulting in the formation of stable compounds. The term “stable” as used herein relates to compounds having sufficient stability to allow preparation and maintain the integrity of the compound for a period of time sufficient to allow the use thereof for the purposes described in detail herein (for example, therapeutic or prophylactic administration to a mammal or use in affinity-chromatographic applications). Typically, such compounds are stable for at least one week at a temperature of 40° C. or less and in absence of moisture or other chemically reactive conditions.

The compounds of the present invention can be used in the form of salts derived from inorganic or organic acids. For example, such acid salts include the following: acetate, adipate, alginate, aspartate, benzoate, benzenesulfonate, bisulfate, citrate, camphorate, camphersulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate.

At least one embodiment also relates to nucleic acids containing as building blocks one or more nucleosides of at least one embodiment. Such nucleic acids can be produced according to methods well-known to those skilled in the art, and in a preferred fashion the nucleic acids of at least one embodiment are constituted of from 2 to 5000, preferably from 10 to 100 nucleoside building blocks, more preferably from 20 to 40 nucleoside building blocks. The nucleic acids of at least one embodiment, as is the case with the nucleosides of at least one embodiment, preferably can be used as antiviral, antibacterial or fungicidal agents, preferably as antiviral agents, and particularly against hepatitis infections. Being antisense nucleic acids, the nucleic acids of at least one embodiment hybridize with the DNA of the virus, thus inhibiting transcription of the viral DNA. In particular, the nucleic acids can be used as agents against hepatitis B, as well as herpes, HIV and others, because degradation thereof by cellular restriction enzymes advantageously is low or difficult.

The synthetic nucleic acids or antisense nucleic acids according to at least one embodiment can be present in the form of a therapeutic composition or formulation which can be used to inhibit DNA replication in a cell and treat human hepatitis infections and concomitant diseases in an animal. They can be used as part of a pharmaceutical composition in combination with a physiologically and/or pharmaceutically tolerable carrier. The properties of the carrier will depend on the route of administration. In addition to synthetic nucleic acid and carrier, such a composition may include diluents, fillers, salts, buffers, stabilizers, solvents and other well-known materials. The pharmaceutical composition of at least one embodiment may also include other active factors and/or substances enhancing the inhibition of HBV expression. For example, combinations of synthetic nucleic acids, each one directed towards a different region of the HBV nucleic acid, can be used in the pharmaceutical compositions of at least one embodiment. Furthermore, the pharmaceutical composition of at least one embodiment may include other chemotherapeutical agents for the treatment of liver carcinomas. Such additional factors and/or substances can be incorporated in the pharmaceutical composition in order to create a synergistic effect together with the synthetic nucleic acids of at least one embodiment or reduce side effects of the synthetic nucleic acids according to at least one embodiment. On the other hand, the synthetic nucleic acids of at least one embodiment can be incorporated in formulations of a particular anti-HBV or anti-cancer factor and/or substance to reduce the side effects of said anti-HBV factor and/or substance.

The pharmaceutical composition of at least one embodiment can be present in the form of a liposome wherein the synthetic nucleic acids of at least one embodiment, in addition to other pharmaceutically tolerable carriers, are combined with amphipathic substances such as lipids, which are present as micelles in one form of aggregation, insoluble monolayers, liquid crystals or lamellar layers present in an aqueous solution. Suitable lipids for a liposomal formulation include—but are not limited to—monoglycerides, diclycerides, sulfatides, lysolecithin, phospholipids, saponins, bile acids and the like. The preparation of such liposomal formulations proceeds in a per se known manner and is well-known to those skilled in the art. Furthermore, the pharmaceutical composition of at least one embodiment may include other lipid carriers such as lipofectamine or cyclodextrins and the like, thereby enhancing the supply of said nucleic acids to the cells, or it may include delayed-release polymers.

At least one embodiment also relates to a pharmaceutical agent comprising at least one nucleoside and/or nucleic acid according to at least one embodiment, optionally together with conventional auxiliaries, preferably carriers, adjuvants and/or vehicles. A pharmaceutical agent in the meaning of at least one embodiment is any agent in the field of medicine, which can be used in the prophylaxis, diagnosis, therapy, follow-up or aftercare of patients who have come in contact with viruses, including hepatitis viruses, in such a way that a pathogenic modification of the overall condition or of the condition of particular parts of the organism could establish at least temporarily. Thus, for example, the pharmaceutical agent in the meaning of at least one embodiment can be a vaccine, an immunotherapeutic or immunoprophylactic agent. The pharmaceutical agent in the meaning of at least one embodiment may comprise the nucleosides or nucleic acids of at least one embodiment and/or an acceptable salt or components thereof. For example, salts of inorganic acids can be concerned, such as phosphoric acid, or salts of organic acids. Furthermore, the salts can be free of carboxyl groups and derived from inorganic bases, such as sodium, potassium, ammonium, calcium or iron hydroxides, or from organic bases such as isopropylamine, trimethylamine, 2-ethylaminoethanol, histidine and others. Examples of liquid carriers are sterile aqueous solutions including no additional materials or active ingredients, such as water, or those including a buffer such as sodium phosphate with a physiological pH value or a physiological salt solution or both, e.g. phosphate-buffered sodium chloride solution. Other liquid carriers may comprise more than just one buffer salt, e.g. sodium and potassium chloride, dextrose, propylene glycol, polyethylene glycol or others.

Liquid compositions of said pharmaceutical agents may additionally comprise a liquid phase, also one excluding water. Examples of such additional liquid phases are glycerol, vegetable oils, organic esters or water-oil emulsions. The pharmaceutical composition or pharmaceutical agent typically includes a content of at least 0.1 wt.-% of nucleosides or nucleic acids of at least one embodiment, relative to the overall pharmaceutical composition. The respective dose or dose range for administering the pharmaceutical agent of at least one embodiment method is in an amount sufficient to achieve the desired prophylactic or therapeutic antiviral effect. The dose should not be selected in such a way that undesirable side effects would dominate. In general, the dose will vary with the age, constitution, sex of a patient, and obviously with respect to the severity of a disease. The individual dose can be adjusted both with respect to the primary disease and with respect to ensuing additional complications. The exact dose can be detected by a person skilled in the art, using well-known means and methods, e.g. by determining the virus titer as a function of the dose or as a function of the vaccination scheme or of the pharmaceutical carriers and the like. Depending on the patient, the dose can be selected individually. For example, a dose of pharmaceutical agent tolerated by a patient can be one where the local level in plasma or in individual organs ranges from 0.1 to 10,000 μM, preferably between 1 and 100 μM. Alternatively, the dose can also be estimated relative to the body weight of the patient. In this event, for example, a typical dose of pharmaceutical agent would be adjusted in a range between 0.1 μg to 100 μg per kg body weight, preferably between 1 and 50 μg/kg. Furthermore, it is also possible to determine the dose with respect to individual organs rather than the overall patient. For example, this would apply to those cases where the pharmaceutical agent of at least one embodiment, incorporated in the respective patient e.g. in a biopolymer, is placed near particular organs by means of surgery. A number of biopolymers capable of liberating the nucleosides or nucleic acids in a desired manner are well-known to those skilled in the art. For example, such a gel may include from 1 to 1000 μg of compounds or pharmaceutical agent of at least one embodiment per ml gel composition, preferably between 5 and 500 μg/ml, and more preferably between 10 and 100 mg/ml. In this event, the therapeutic agent will be administered in the form of a solid, gel-like or liquid composition.

In a preferred fashion the pharmaceutical agent may also include one or more additional agents from the group of antiviral, fungicidal or antibacterial agents and/or immunostimulators. In a preferred fashion the antiviral agent concerns protease inhibitors and/or reverse transcriptase inhibitors. The immunostimulators are preferably bropirimine, anti-human alpha-interferon antibodies, IL-2, GM-CSF, interferons, diethyl dithiocarbamate, tumor necrosis factors, naltrexone, tuscarasol and/or rEPO.

In another preferred embodiment of at least one embodiment the carriers are selected from the group comprising fillers, diluents, binders, humectants, disintegrants, dissolution retarders, absorption enhancers, wetting agents, adsorbents and/or lubricants.

The fillers and diluents are preferably starches, lactose, cane-sugar, glucose, mannitol and silica, the binder is preferably carboxymethylcellulose, alginate, gelatin, polyvinylpyrrolidone, the humectant is preferably glycerol, the disintegrant is preferably agar, calcium carbonate and sodium carbonate, the dissolution retarder is preferably paraffin, and the absorption enhancer is preferably a quaternary ammonium compound, the wetting agent is preferably cetyl alcohol and glycerol monostearate, the adsorbent is preferably kaolin and bentonite, and the lubricant is preferably talc, calcium and magnesium stearates and solid polyethylene glycols, or mixtures of the materials mentioned above.

At least one embodiment also relates to vectors, cells and/or organisms having a nucleoside of at least one embodiment, a nucleic acid of at least one embodiment and/or a pharmaceutical agent of at least one embodiment.

At least one embodiment also relates to the use of the nucleosides of at least one embodiment, the nucleic acids of at least one embodiment and/or the pharmaceutical agent of at least one embodiment in the prophylaxis or therapy of a viral, bacterial, fungicidal and/or parasitic infection or of cancer. For example, it is well-known to those skilled in the art that viruses can induce various tumors. Using the compounds of at least one embodiment, such tumors can be prevented prophylactically or treated therapeutically. Obviously, the structures of at least one embodiment can also be utilized in an anticancer combination therapy, for example. Those skilled in the art are also familiar with the fact that, in addition to viruses, bacteria associated with viral diseases or appearing by themselves represent a medical problem. Numerous bacteria have resistance to well-known antibacterial agents. The compounds of at least one embodiment can be used in the prophylaxis and treatment of bacterial infections as well. Furthermore, the compounds of at least one embodiment can be used in the production of drugs for the treatment and prophylaxis of bacterial infections. In a preferred fashion the bacteria can be those from the genuses Escherichia coli, Salmonella spp., Shigella flexneri, Citrobacter freundii, Klebsiella pneumoniae, Vibrio spp., Haemophilus influenzae, Yersinia enterolitica, Pasturella haemolytica, and Proteus spp.

In another preferred embodiment at least one embodiment relates to the use of the compounds of at least one embodiment to prevent incorporation of other nucleosides during transcription in a growing DNA chain, prevent formation of a DNA-RNA hybrid, separate a base pair, or in competitive inhibition of a growing DNA chain.

In another preferred embodiment of at least one embodiment, the compounds of at least one embodiment are used in a prophylactic or therapeutic treatment of viral diseases associated with one of the following viruses or a combination thereof: hepatitis virus, HIV, bovine immunodeficiency virus, human T cell leukemia virus, feline immunodeficiency virus, caprine arthritis-encephalitis virus, equine infectious anemia virus, ovine Maedi-Visna virus, Visna-Lenti virus and others. In a preferred fashion, DNA viruses are treated. Those skilled in the art are familiar with the fact that the incidence of such viral infections can be combined with bacterial, fungicidal, parasitic or other infections.

Such use is particularly preferred in those cases where the hepatitis virus is a hepatitis B or a hepatitis D virus. In a likewise particularly preferred fashion the pharmaceutical agent of at least one embodiment comprises inhibitors of HBV DNA polymerase. Obviously, the pharmaceutical agent for treatment, especially of hepatitis B, may include further effective anti-HBV agents, preferably PMEA (adefovir-dipivoxil), famciclovir, penciclovir, diaminopurine-dioxolane (DAPD), clevudine (L-FMAU), entecavir, interferon or thymosin α1 and/or inhibitors of nucleocapsid formation, particularly heteroarylpyrimidines.

In a likewise preferred fashion the agents are pegylated.

Moreover, it is particularly preferred that the agent includes additional agents capable of eliminating the function of cellular proteins essential to HBV growth.

In a likewise particularly preferred fashion the above agent includes agents against viruses resistant to lamivudine or other cytosine nucleosides, such as emtricitabine (L-FTC), L-ddC or L-ddeC.

In a preferred fashion the agent can also be employed against liver carcinoma diseases triggered by chronic hepatitis, particularly by HBV.

In a likewise preferred fashion the β-L-nucleosides enhance the effect of other pharmaceutical agents in a non-additive, additive or synergistic fashion, increase the therapeutic index and/or reduce the risk of toxicity inherent in the respective compounds.

A preferred HIV in the meaning of at least one embodiment is HIV-1 with the subtypes A to J (HIV-1 group M) in accordance with the prior art subtype classification and the distantly related HIV-O (HIV-1 group O). Preferred main subtypes are 1A, 1B, 1C and 1D. The subtypes 1E, 1G and 1H are closely related to HIV-1A and likewise preferred. The preferred HIV-1A and 1C, as well as 1B and 1D show homology with respect to each other. The likewise preferred HIV-O is more heterogeneous than HIV-1 in particular virus isolates. Classification into subtypes is not possible. Also preferred is HIV-2 which can be classified into the subtypes A to E. It has milder pathogenicity compared to HIV-1 and has therefore spread more slowly. The genetic variability results in changes in the external coat proteins. The influence on cytotropism, as well as the question to what extent this is accompanied by varying transmission probabilities have not been clarified sufficiently. Likewise preferred is treatment of double infections with different subtypes (e.g. B and E).

In a preferred embodiment, the nucleosides are used in combination with 3-deazauridine. Combined use may involve simultaneous or time-shifted administration. Such combined administration can be effected in a combined agent, for example.

For example, the combined agent in the meaning of at least one embodiment can be such in nature that nucleosides of at least one embodiment and 3-deazauridine are included together in a solution or solid, e.g. in a tablet. In this event, the ratio of nucleosides of at least one embodiment and 3-deazauridine may vary freely. A ratio of nucleosides of at least one embodiment and 3-deazauridine ranging from 1:10,000 to 10,000:1 is preferred. The ratio of nucleosides of at least one embodiment and 3-deazauridine may vary within this range, depending on the desired application. Of course, said at least two components—nucleosides of at least one embodiment and 3-deazauridine—can also be incorporated together in a solution or solid in such a way that release thereof will proceed in a time-shifted fashion. However, the combined agent in the meaning of at least one embodiment may also be constituted of two separate solutions or two separate solids, one solution or solid essentially comprising 3-deazauridine and the other solution or solid essentially comprising the nucleosides of at least one embodiment. The two solutions or solids can be associated with a common carrier or with separate carriers. For example, the two solutions and/or the two solids can be present in a capsule as common carrier. Such a formulation of the combined agent of at least one embodiment is advantageous in those cases where administration of the nucleosides of at least one embodiment and 3-deazauridine is to proceed in a time-shifted manner. That is, the organism is initially contacted with nucleosides of at least one embodiment, e.g. by infusion or oral administration, to be contacted with the other component of the combined agent in a time-shifted manner. Of course, it is also possible to provide the combined agent by means of conventional pharmaceutical-technical methods and procedures in such a way that the organism is initially contacted with 3-deazauridine and subsequently with the nucleosides of at least one embodiment. Hence, the organism is contacted sequentially with the components of the combined agent. The time period between administration of the two components of the combined agent of at least one embodiment or the initial release of nucleosides of at least one embodiment or 3-deazauridine depends on the age, sex, overall constitution of the patient, the disease, or other parameters which can be determined by the attending physician using prior tests, for example.

In a particularly preferred embodiment of at least one embodiment the compounds of at least one embodiment are used as a prodrug, as feed additive and/or as drinking water additive, the use as feed additive and/or drinking water additive being preferred in veterinary medicine.

In a particularly preferred fashion the compounds of at least one embodiment are used as prodrug. The utilization of endocytosis for the cellular uptake of active substances comprising polar compounds is highly effective for some, particularly long-lived substances, but is very difficult to transfer to more general uses. One alternative is the prodrug concept generally known to those skilled in the art. By definition, a prodrug includes its active substance in the form of a non-active precursor metabolite. It is possible to distinguish between carrier prodrug systems, some of them being multi-component ones, and biotransformation systems. The latter include the active substance in a form requiring chemical or biological metabolization. Such prodrug systems are well-known to those skilled in the art, e.g. valacyclovir as a precursor of acyclovir, or others. Carrier prodrug systems include the active substance as such, bound to a masking group which can be cleaved off by a preferably simple controllable mechanism. The inventive function of masking groups in the nucleosides of at least one embodiment is neutralization of the negative charge on the phosphate residue for improved reception by cells. When using the nucleosides of at least one embodiment together with a masking group, the latter may also influence other pharmacological parameters, such as oral bioavailability, distribution in tissue, pharmacokinetics, as well as stability to non-specific phosphatases. In addition, delayed release of the active substance may entail a depot effect. Furthermore, modified metabolization may occur, thereby achieving higher efficiency of the active substance or organ specificity. In the event of a prodrug formulation, the masking group, or a linker group binding the masking group to the active substance, is selected in such a way that the nucleoside prodrug has sufficient hydrophilicity to be dissolved in the blood serum, sufficient chemical and enzymatic stability to reach the site of action, and hydrophilicity suitable for diffusion-controlled membrane transport. Furthermore, it should permit chemical or enzymatic liberation of the active substance within a reasonable period of time and, of course, the liberated auxiliary components should not be toxic. In the meaning of at least one embodiment, however, the nucleoside with no mask or no linker and no mask can also be understood as prodrug because the structure inhibiting viral DNA polymerase is a high-energy triphosphate which initially must be provided via enzymatic and biochemical processes from the incorporated nucleoside in the cell.

In another particularly preferred embodiment, the compounds of are formulated as a gel, powder, tablet, sustained-release tablet, premix, emulsion, brew-up formulation, drops, concentrate, granulate, syrup, pellet, bolus, capsule, aerosol, spray and/or inhalant and/or used in this form. The tablets, coated tablets, capsules, pills and granulates can be provided with conventional coatings and envelopes optionally including opacification agents, and can be composed such that release of the active substance(s) takes place only or preferably in a particular area of the intestinal tract, optionally in a delayed fashion, to which end polymer substances and waxes can be used as embedding materials.

Preferably, the drugs of the present invention can be used in oral administration in any orally tolerable dosage form, including capsules, tablets and aqueous suspensions and solutions, without being restricted thereto. In case of tablets for oral application, carriers frequently used include lactose and corn starch. Typically, lubricants such as magnesium stearate can be added. For oral administration in the form of capsules, diluents that can be used include lactose and dried corn starch. In oral administration of aqueous suspensions the active substance is combined with emulsifiers and suspending agents. Also, particular sweeteners and/or flavors and/or coloring agents can be added, if desired.

The active substance(s) can also be present in micro-encapsulated form, optionally with one or more of the above-specified carrier materials.

In addition to the active substance(s), suppositories may include conventional water-soluble or water-insoluble carriers such as polyethylene glycols, fats, e.g. cocoa fat and higher esters (for example, C₁₄ alcohols with C₁₆ fatty acids) or mixtures of these substances.

In addition to the active substance(s), ointments, pastes, creams and gels may include conventional carriers such as animal and vegetable fats, waxes, paraffins, starch, tragacanth, cellulose derivatives, polyethylene glycols, silicones, bentonites, silica, talc and zinc oxide or mixtures of these substances.

In addition to the active substance(s), powders and sprays may include conventional carriers such as lactose, talc, silica, aluminum hydroxide, calcium silicate and polyamide powder or mixtures of these substances. In addition, sprays may include conventional propellants such as chlorofluorohydrocarbons.

In addition to the active substance(s), solutions and emulsions may include conventional carriers such as solvents, solubilizers, and emulsifiers such as water, ethyl alcohol, isopropyl alcohol, ethyl carbonate, ethyl acetate, benzyl alcohol, benzyl benzoate, propylene glycol, 1,3-butylene glycol, dimethylformamide, oils, especially cotton seed oil, peanut oil, corn oil, olive oil, castor oil and sesame oil, glycerol, glycerol formal, tetrahydrofurfuryl alcohol, polyethylene glycols, and fatty esters of sorbitan, or mixtures of these substances. For parenteral application, the solutions and emulsions may also be present in a sterile and blood-isotonic form.

In addition to the active substance(s), suspensions may include conventional carriers such as liquid diluents, e.g. water, ethyl alcohol, propylene glycol, suspending agents, e.g. ethoxylated isostearyl alcohols, polyoxyethylenesorbitol and sorbitan esters, microcrystalline cellulose, aluminum metahydroxide, bentonite, agar, and tragacanth, or mixtures of these substances.

The drugs can be present in the form of a sterile injectable formulation, e.g. as a sterile injectable aqueous or oily suspension. Such a suspension can also be formulated by means of methods known in the art, using suitable dispersing or wetting agents (such as Tween 80) and suspending agents. The sterile injectable formulation can also be a sterile injectable solution or suspension in a non-toxic, parenterally tolerable diluent or solvent, e.g. a solution in 1,3-butanediol. Tolerable vehicles and solvents that can be used include mannitol, water, Ringer's solution, and isotonic sodium chloride solution. Furthermore, sterile, non-volatile oils are conventionally used as solvents or suspending medium. Any mild non-volatile oil, including synthetic mono- or diglycerides, can be used for this purpose. Fatty acids such as oleic acid and glyceride derivatives thereof can be used in the production of injection agents, e.g. natural pharmaceutically tolerable oils such as olive oil or castor oil, especially in their polyoxyethylated forms. Such oil solutions or suspensions may also include a long-chain alcohol, such as Ph.Helv., or a similar alcohol as diluent or dispersant.

The above-mentioned formulation forms may also include colorants, preservatives, as well as odor- and taste-improving additives, e.g. peppermint oil and eucalyptus oil, and sweeteners, e.g. saccharine. Preferably, the active substances of formula (I) and (II) should be present in the above-mentioned pharmaceutical preparations at a concentration of about 0.1 to 99.5 wt.-%, more preferably about 0.5 to 95 wt.-% of the overall mixture.

In addition to the compounds of formula (I) and (II), the above-mentioned pharmaceutical preparations may include further pharmaceutical active substances. The production of the pharmaceutical preparations specified above proceeds in a usual manner according to well-known methods, e.g. by mixing the active substance(s) with the carrier material(s).

The above-mentioned preparations can be applied in humans and animals on an oral, rectal, parenteral (intravenous, intramuscular, subcutaneous), intracisternal, intravaginal, intraperitoneal route, locally (powders, ointment, drops) and used in the therapy of infections in hollow areas and body cavities. Injection solutions, solutions and suspensions for oral therapy, gels, brew-up formulations, emulsions, ointments or drops are possible as suitable preparations. For local therapy, ophthalmic and dermatological formulations, silver and other salts, ear drops, eye ointments, powders or solutions can be used. With animals, ingestion can be effected via feed or drinking water in suitable formulations. Furthermore, gels, powders, tablets, sustained-release tablets, premixes, concentrates, granulates, pellets, boli, capsules, aerosols, sprays, inhalants can be used in humans and animals. Moreover, the compounds of at least one embodiment can be incorporated in other carrier materials such as plastics (plastic chains for local therapy), collagen or bone cement.

In another preferred embodiment the compounds, i.e., the nucleosides of at least one embodiment, the nucleic acids of at least one embodiment, the inventive pharmaceutical agents or vectors, cells and organisms, are incorporated in a preparation at a concentration of 0.1 to 99.5, preferably 0.5 to 95, and more preferably 20 to 80 wt.-%. That is, the compounds of at least one embodiment are present in the above-specified pharmaceutical formulations, e.g. tablets, pills, granulates and others, at a concentration of preferably 0.1 to 99.5 wt.-% of the overall mixture. Those skilled in the art will be aware of the fact that the amount of active substance, i.e., the amount of an inventive compound combined with the carrier materials to produce a single dosage form, will vary depending on the host to be treated and on the particular type of administration. Once the condition of a host or patient has improved, the proportion of active compound in the preparation can be modified so as to obtain a maintenance dose. Depending on the symptoms, the dose or frequency of administration or both can subsequently be reduced to a level where the improved condition is retained. Once the symptoms have been alleviated to the desired level, the treatment should be terminated. However, patients may require an intermittent treatment on a long-term basis if any symptoms of the disease should recur. Accordingly, the proportion of the compounds, i.e. their concentration, in the overall mixture of the pharmaceutical preparation, as well as the composition or combination thereof, is variable and can be modified and adapted by a person of specialized knowledge in the art.

Those skilled in the art will be aware of the fact that the compounds of at least one embodiment can be contacted with an organism, preferably a human or an animal, on various routes. Furthermore, a person skilled in the art will also be familiar with the fact that the pharmaceutical agents in particular can be applied at varying dosages. Application should be effected in such a way that a viral disease is combatted as effectively as possible or the onset of such a disease is prevented by a prophylactic administration. Concentration and type of application can be determined by a person skilled in the art using routine tests. Preferred applications of the compounds of at least one embodiment are oral application in the form of powders, tablets, juice, drops, capsules or the like, rectal application in the form of suppositories, solutions and the like, parenteral application in the form of injections, infusions and solutions, inhalation of vapors, aerosols and powders and pads, and local application in the form of ointments, pads, dressings, lavages and the like. Contacting with the compounds according to at least one embodiment is preferably effected in a prophylactic or therapeutic fashion. In prophylactic administration, an infection with the above-mentioned viruses is to be prevented at least in such a way that, following invasion of single viruses, e.g. into a wound, further growth thereof is massively reduced or viruses having invaded are destroyed virtually completely. In therapeutic contacting, a manifest infection of the patient is already existing, and the viruses already present in the body are either to be destroyed or inhibited in their growth. Other forms of application preferred for this purpose are e.g. subcutaneous, sublingual, intravenous, intramuscular, intraperitoneal and/or topical ones.

For example, the suitability of the selected form of application, of the dose, application regimen, selection of adjuvant and the like can be determined by taking serum aliquots from the patient, i.e., human or animal, and testing for the presence of viruses, i.e., determining the virus titer, in the course of the treatment procedure.

Alternatively or concomitantly, the condition of the liver, but also, the amount of T cells or other cells of the immune system can be determined in a conventional manner so as to obtain a general survey on the immunological constitution of the patient and, in particular, the constitution of organs important to the metabolism, particularly of the liver. Additionally, the clinical condition of the patient can be observed for the desired effect, especially the anti-infectious, preferably antiviral effect. As set forth above, especially hepatitis, but also HIV or other diseases can be associated with other e.g. bacterial or fungicidal infections or tumor diseases, for which reason additional clinical co-monitoring of the course of such concomitant infections or tumor diseases is also possible. Where insufficient therapeutic effectiveness is achieved, the patient can be subjected to further treatment using the agents of at least one embodiment, optionally modified with other well-known medicaments expected to bring about an improvement of the overall constitution. Obviously, it is also possible to modify the carriers or vehicles of the pharmaceutical agent or to vary the route of administration. In addition to oral ingestion, e.g. intramuscular or subcutaneous injections or injections into the blood vessels can be envisaged as other preferred routes of therapeutic administration of the compounds according to at least one embodiment. At the same time, supply via catheters or surgical tubes can also be used.

In addition to the above-specified concentrations during use of the compounds of at least one embodiment, the compounds in a preferred embodiment can be employed in a total amount of 0.05 to 500 mg/kg body weight per 24 hours, preferably 5 to 100 mg/kg body weight. Advantageously, this is a therapeutic quantity which is used to prevent or improve the symptoms of a disorder or of a responsive, pathologically physiological condition. The amount administered is sufficient to prevent or inhibit infection or spreading of an infectious agent such as hepatitis B or HIV in the recipient. With respect to their prophylactic or therapeutic potential, the effect of the compounds of at least one embodiment on the above-mentioned viruses is seen e.g. as an inhibition of the viral infection, inhibition of syncytium formation, inhibition of fusion between virus and target membrane, as a reduction or stabilization of the viral growth rate in an organism, or in another way. For example, the therapeutic effect can be such that, as a desirable side effect, particular antiviral medicaments are improved in their effect or, by reducing the dose, the number of side effects of these medicaments will be reduced as a result of applying the compounds of at least one embodiment. Of course, the therapeutic effect also encompasses direct action on the viruses in a host. That is, however, the effect of the compounds of at least one embodiment is not restricted to eliminating viruses, but rather comprises the entire spectrum of advantageous effects in prophylaxis and therapy. Obviously, the dose will depend on the age, health and weight of the recipient, degree of the disease, type of required simultaneous treatment, frequency of the treatment and type of the desired effects and side-effects. The daily dose of 0.05 to 500 mg/kg body weight can be applied as a single dose or multiple doses in order to furnish the desired results. The dose levels per day can be used in prevention and treatment of a viral infection, including hepatitis B infection. More specifically, pharmaceutical agents are typically used in about 1 to 7 administrations per day, or alternatively or additionally as a continuous infusion. Such administrations can be applied as a chronic or acute therapy. Of course, the amounts of active substance that are combined with the carrier materials to produce a single dosage form may vary depending on the host to be treated and on the particular type of administration. In a preferred fashion, the daily dose is distributed over 2 to 5 applications, with 1 to 2 tablets including an active substance content of 0.05 to 500 mg/kg body weight being administered in each application. Of course, it is also possible to select a higher content of active substance, e.g. up to a concentration of 5000 mg/kg. The tablets can also be sustained-release tablets, in which case the number of applications per day is reduced to 1 to 3. The active substance content of sustained-release tablets can be from 3 to 3000 mg. If the active substance—as set forth above—is administered by injection, the host is preferably contacted 1 to 8 times per day with the compounds of at least one embodiment or by using continuous infusion, in which case quantities of from 1 to 4000 mg per day are preferred. The preferred total amounts per day were found advantageous both in human and veterinary medicine. It may become necessary to deviate from the above-mentioned dosages, and this depends on the nature and body weight of the host to be treated, the type and severity of the disease, the type of formulation and application of the drug, and on the time period or interval during which the administration takes place. Thus, it may be preferred in some cases to contact the organism with less than the amounts mentioned above, while in other cases the amount of active substance specified above has to be surpassed. A person of specialized knowledge in the art can determine the optimum dosages required in each case and the type of application of the active substances.

In another particularly preferred embodiment of at least one embodiment the compounds of at least one embodiment, i.e., the nucleoside, the nucleic acid, the pharmaceutical agent, the vector, the cells and/or organism, are used in a single administration of from 1 to 80, especially from 3 to 30 mg/kg body weight. In the same way as the total amount per day, the amount of a single dose per application can be varied by a person of specialized knowledge in the art. Similarly, the compounds used according to at least one embodiment can be employed in veterinary medicine with the above-mentioned single concentrations and formulations together with the feed or feed formulations or drinking water. A single dose preferably includes that amount of active substance which is administered in one application and normally corresponds to one whole, one half daily dose or one third or one quarter of a daily dose. Accordingly, the dosage units may preferably include 1, 2, 3 or 4 or more single doses or 0.5, 0.3 or 0.25 single doses. In a preferred fashion, the daily dose of the compounds according to at least one embodiment is distributed over 2 to 10 applications, preferably 2 to 7, and more preferably 3 to 5 applications. Of course, continuous infusion of the agents according to at least one embodiment is also possible.

In a particularly preferred embodiment, 1 to 2 tablets are administered in each oral application of the compounds of at least one embodiment. The tablets according to at least one embodiment can be provided with coatings and envelopes well-known to those skilled in the art or can be composed in a way so as to release the active substance(s) only in preferred, particular regions of the host.

In another preferred embodiment of at least one embodiment the compounds according to at least one embodiment can be employed together with at least one other well-known pharmaceutical agent. That is to say, the compounds of at least one embodiment can be used in a prophylactic or therapeutic combination in connection with well-known drugs. Such combinations can be administered together, e.g. in an integrated pharmaceutical formulation, or separately, e.g. in the form of a combination of tablets, injection or other medications administered simultaneously or at different times, with the aim of achieving the desired prophylactic or therapeutic effect. These well-known agents can be agents which enhance the effect of the nucleosides according to at least one embodiment. In the antibacterial sector, in particular, it was found that a wide variety of antibiotics improve the effect of nucleosides. This includes agents such as benzylpyrimidines, pyrimidines, sulfoamides, rifampicin, tobramycin, fusidinic acid, clindamycin, chloramphenicol and erythromycin. Accordingly, another embodiment of at least one embodiment relates to a combination wherein the second agent is least one of the above-mentioned antiviral or antibacterial agents or classes of agents. It should also be noted that the compounds of at least one embodiment and combinations can also be used in connection with immune-modulating treatments and therapies.

Typically, there is an optimum ratio of compound(s) of at least one embodiment with respect to each other and/or with respect to other therapeutic or effect-enhancing agents (such as transport inhibitors, metabolic inhibitors, inhibitors of renal excretion or glucuronidation, such as probenecid, acetaminophen, aspirin, lorazepan, cimetidine, ranitidine, colifibrate, indomethacin, ketoprofen, naproxen etc.) where the active substances are present at an optimum ratio. Optimum ratio is defined as the ratio of compound(s) of at least one embodiment to other therapeutic agent(s) where the overall therapeutic effect is greater than the sum of the effects of the individual therapeutic agents. In general, the optimum ratio is found when the agents are present at a ratio of from 10:1 to 1:10, from 20:1 to 1:20, from 100:1 to 1:100 and from 500:1 to 1:500. In some cases, an exceedingly small amount of a therapeutic agent will be sufficient to increase the effect of one or more other agents. In addition, the use of the compounds of at least one embodiment in combinations is particularly beneficial in order to reduce the risk of developing resistance. Of course, the compounds of at least one embodiment, such as nucleosides or nucleic acids, can be used in combination with other well-known antiviral agents. Such agents are well-known to those skilled in the art. Accordingly, the compounds of at least one embodiment can be administered together with all conventional agents, especially other drugs, available for use particularly in connection with hepatitis drugs, either as a single drug or in a combination of drugs. They can be administered alone or in combination with same.

In a preferred fashion the compounds of at least one embodiment are administered together with said other well-known pharmaceutical agents at a ratio of about 0.005 to 1. Preferably, the compounds of at least one embodiment are administered particularly together with virus-inhibiting agents at a ratio of from 0.05 to about 0.5 parts and up to about 1 part of said known agents. In this event, tumor-inhibiting or antibacterial agents can be concerned. The pharmaceutical composition can be present in substance or as an aqueous solution together with other materials such as preservatives, buffer substances, agents to adjust the osmolarity of the solution, and so forth.

At least one embodiment also relates to the use of the nucleic acids of at least one embodiment as antisense nucleic acids, particularly in an antiviral therapy. Those skilled in the art are familiar with the fact that nucleic acids can be used as antisense nucleic acids. In a preferred fashion the nucleic acid of at least one embodiment serves to prevent hybridization of the RNA during translation, and this proceeds via hybridization of the viral RNA with the nucleic acids according to at least one embodiment. More specifically, the nucleic acids of at least one embodiment can be used as agents against hepatitis B because degradation thereof by cellular restriction enzymes is absent or difficult. In general, the nucleic acid of at least one embodiment hybridizes with the DNA of the hepatitis B virus, thereby not only impeding translation, but also transcription into viral DNA.

The nucleosides and nucleic acids according to at least one embodiment can be used in the production of pharmaceutical agents. Thus, the teaching of at least one embodiment may also relate to a method for the treatment of a viral, bacterial, fungicidal and/or parasitic infection or of cancer, in which method the nucleosides and/or nucleic acids of at least one embodiment are contacted with an organism.

Treatment in the meaning of at least one embodiment includes both prophylactic and therapeutic treatment. In a preferred fashion the compounds of at least one embodiment can be used to protect organisms, especially human patients, from viral infection during a particular incident, such as delivery, or for a prolonged period of time, in a country where high risk of hepatitis B infection exists. In such cases, the compounds of at least one embodiment can be used alone or together with other prophylactic agents or other antiviral agents enhancing the efficacy of the respective agent. Preferably following oral application, the nucleosides of at least one embodiment advantageously can undergo easy absorption into the bloodstream of mammals, especially human mammals. Advantageously, the compounds exhibit good water solubility and consistent oral availability. In particular, it is said good oral availability that makes the compounds of at least one embodiment excellent agents for orally administered cures of treatment and prevention against viral infection, especially hepatitis B infection. Of course, the compounds of at least one embodiment not only are orally bioavailable, but advantageously have also a high therapeutic index which, in particular, is a measure of toxicity versus antiviral effect. Accordingly, the compounds of at least one embodiment are more effective at lower dose levels compared to selected well-known antiviral agents, avoiding the toxic effect associated with these medical substances. The potential of the compounds of at least one embodiment of being released at doses far exceeding their active antiviral range is particularly advantageous in slowing down or preventing possible development of resistant variants. During a prophylactic treatment, in particular, the compounds of at least one embodiment can be used in a healthy, but also in a virally infected, especially in a hepatitis B virus infected patient, either as a single agent or together with other antiviral agents preferably impairing the replication cycle of hepatitis viruses. The use of the compounds of at least one embodiment in prophylaxis and therapy proceeds in a way well-known to those skilled in the art. In those cases where the method of treating a viral infection with the nucleosides of at least one embodiment represents a combination therapy, each agent used, i.e., both the well-known compounds and the compounds of at least one embodiment, has an additive, non-additive or synergistic effect in inhibiting virus replication, because action of each agent at a different site of replication of the viruses advantageously can be envisaged. Advantageously, the method of such combination therapies can also reduce the dosage of a conventional antiviral agent which, in comparison (when administering the agent alone), would be required for a desired therapeutic or prophylactic effect. Such combinations in the method of at least one embodiment for the treatment of viral diseases can reduce or eliminate the side effects of conventional therapies using single antiviral agents, and such combinations advantageously do not impair but rather synergistically increase the antiviral effect of these agents. These combinations reduce the potential of resistance to therapy using single agents, while advantageously minimizing the toxicity associated therewith. These combinations can also increase the efficacy of conventional agents without increasing the toxicity associated therewith. In a particularly preferred fashion the compounds according to this invention, together with other antiviral or antibacterial or fungicidal agents, prevent replication of the genetic material of viruses in an additive or synergistic manner. Inter alia, preferred combination therapies include the administration of a compound of at least one embodiment together with ACTddi, ddC, d4T, 3TC or a combination thereof. Of course, administration together with other nucleoside derivatives or viral reverse transcriptase inhibitors or protease inhibitors may also be preferred in the method of at least one embodiment or in the use according to at least one embodiment. Joint administration of the compounds of at least one embodiment and viral reverse transcriptase inhibitors or aspartyl protease inhibitors shows an additive or synergistic effect, thereby preventing, essentially reducing or completely eliminating virus replication or infection or both, or symptoms associated therewith. Administration of a combination of agents can be preferred over administration of single agents. The compounds of at least one embodiment can also be used together with immunomodulators or immunostimulators; preferred immunomodulators or immunostimulators are: bropirimine, anti-human α-interferon antibodies, IL-2, GM-CSF, interferon α, diethyl dithiocarbamate, tumor necrosis factor, naltrexone, tuscarasol, rEPO and antibiotics such as pentamidine isethionate, but also agents preventing or combatting malignant tumors associated with viral diseases. In the method for the treatment of viral, bacterial, fungicidal and/or parasitic infections or of cancer, the compounds of at least one embodiment—as set forth above—can be administered together with tolerable carriers, adjuvants or vehicles. Pharmaceutically tolerable carriers, adjuvants and vehicles that can be used in the drugs of this invention include ion exchangers, aluminum oxide, aluminum stearate, lecithin, self-emulsifying drug delivery systems (SEDDS), such as d-α-tocopherol-polyethylene glycol 1000 succinate, or other similar polymer delivery matrices, serum proteins such as human serum albumin, buffer substances such as phosphates, glycine, sorbic acids, potassium sorbate, partial glyceride mixtures of saturated vegetable fatty acids, water, salts or electrolytes such as protamin sulfate, disodium hydrogen phosphate, potassium hydrogen phosphate, sodium chloride, zinc salts, colloidal silicon dioxide, magnesium trisilicates, polyvinylpyrrolidone, materials on cellulose basis, polyethylene glycol, sodium carboxymethylcellulose, polyacrylates, waxes, polyethylene-polyoxypropylene block polymers, polyethylene glycol, and wool fat, but are not restricted thereto. Cyclodextrins such as α-, β-, and γ-cyclodextrin or chemically modified derivatives such as hydroxyalkylcyclodextrins, including 2- and 3-hydroxypropyl-α-cyclodextrins or other solubilized derivatives, can also be used with advantage in order to enhance delivery of the compounds according to at least one embodiment. In the context with this method, the compounds of at least one embodiment can be administered orally, parenterally, via inhalation spray, topically, rectally, nasally, buccally, vaginally, or via implanted reservoirs. Oral administration or administration via injection is a preferred form of contacting. The drugs of this invention may include any conventional non-toxic, pharmaceutically tolerable carriers, adjuvants or vehicles. In some cases, the pH value of the formulation can be adjusted using pharmaceutically tolerable acids, bases or buffers in order to increase the stability of the formulated compound or delivery form thereof. The term parenteral, as used herein, includes subcutaneous, intracutaneous, intravenous, intramuscular, intra-articular, intrasynovial, intrasternal, intrathecal, intralesional and intracranial injection or infusion procedures as a form of contacting.

At least one embodiment also relates to a kit comprising the compounds of at least one embodiment, optionally together with information on how to combine the contents of the kit. The information for combining the contents of the kit relates to the use of said kit in the prophylaxis and/or therapy of diseases, particularly viral diseases. For example, the information may also concern a therapeutic scheme, i.e., a concrete injection or application scheme, the dose to be administered, or other.

The nucleoside analogs of at least one embodiment have many advantages. In the course of their individual development, human and animal organisms must cope with numerous pathogens. For example, these pathogens can be fungi, bacteria, but also viruses, in particular. Each year, millions of people and economically useful animals develop a viral disease, and a large number of such infections are accompanied by significant health impairments. Untreated for a prolonged period of time, diseases with human immunodeficiency virus and hepatitis viruses can be fatal.

The viruses an organism has to cope with strongly differ in their infectious potential. Highly infectious viruses include hepatitis B virus (HBV) which may cause inflammations of the liver, regularly accompanied by liver cell damage, and such liver damage can develop up to a liver tumor in chronic courses with selected viruses, such as hepatitis viruses B, C and D.

To allow successful combatting of viruses in a host organism, e.g. in a human or in a farm or domestic animal, the prior art has developed various antiviral therapies. A large number of these therapies are chemotherapies intended to prevent replication of pathogenic viruses in a host cell. Various phases of replication, such as adsorption, penetration, translation, transcription of the viral genes, replication of nucleic acids, as well as assembly of virus particles, are possible as targets of attack for the so-called virustatic agents used to this end. Virus adsorption inhibitors interact with cationic regions of the viral coat protein, thereby preventing association with receptors of the potential host cell. In contrast to the adsorption inhibitors, the inhibitors of virus cell fusion do not act as early as to prevent binding, but rather act at a later stage to prevent fusion with the host cell to form a common membrane. Another way would be inhibition of penetration with liberation of the viral genome, as has been described in the prior art, e.g. for Picorna viruses. Furthermore, it is possible to block the transcription and protein biosynthesis of viruses. Methods of inhibiting viral DNA polymerase have also been described in the prior art. The inhibition of viral DNA polymerase has been disclosed in the prior art particularly for herpes viruses. The DNA polymerase of herpes viruses assumes various functions. Among other things, it is responsible for the introduction of the viral genetic information into the host cell genome, for RNA-dependent DNA synthesis, for DNA-dependent DNA synthesis, and has additional functions. A large number of presently known, successfully applied antiviral compounds are nucleoside-analogous substances which, however, are limited in their antiviral activity to herpes viruses in particular.

As the above-mentioned strategies are successful in herpes viruses, in particular, and allow application to other viruses with less success in some cases, it has been necessary to develop different therapies for each particular group of viruses. Thus, for example, vaccines produced by genetic engineering have been available for years for the treatment of hepatitis B; however, they fail to be helpful in individuals already infected and exert significant influence on the above-mentioned chronic course of said disease. The nucleosides of at least one embodiment avoid the above-specified drawbacks of the prior art.

Without intending to be limiting, at least one embodiment will be explained in more detail with reference to the following examples.

### EXAMPLES

### 1. Synthesis of β-L-5-methyldeoxycytidine (β-L-MetCdR)

### 1.1 1-(3,5-Di-O-acetyl-2-deoxy-β-L-ribofuranosyl)thymine

L-Thymidine (7.65 mmol, 1.85 g) is dissolved in dry pyridine (15 ml). The mixture is added with acetic anhydride (2.9 ml, 30.6 mmol) and maintained at room temperature for 12 hours with stirring. Subsequently, the solvent is removed in vacuum. The residue is dissolved in chloroform, and the resulting solution is washed twice with saturated sodium hydrogen carbonate solution and subsequently with water. The organic phase is dried with sodium sulfate. Following stripping of the solvent in vacuum, the resulting residue is purified by means of column chromatography on silica gel, using chloroform/methanol (99/1, v/v) as eluent. The corresponding fractions yield 2.4 g of the di-O-acetyl compound in the form of a white foam.

### 1.2 1-(2-Deoxy-β-L-ribofuranosyl)-5-methylcytosine (β-L-5-methyldeoxycytidine, β-L-MetCdR)

1-(3,5-Di-O-acetyl-2-deoxy-β-L-ribofuranosyl)thymine (2.4 g, 7.36 mmol) is dissolved in anhydrous pyridine (50 ml) and added with 1H-1,2,4-triazole (1.02 g, 11.14 mmol) and chlorophenyl dichlorophosphate (1.8 ml, 11.14 mmol). The reaction mixture is maintained at room temperature for four days with stirring. Thereafter, the pyridine is removed in vacuum, and the residue is dissolved in dioxane/25% NH₄OH (3/1, v/v) and stored at room temperature overnight. The solvent is removed in vacuum, and the resulting dark residue is dissolved in water (40 ml) and applied on a column (2×20 cm) containing DOWEX 50 WX 8 (H⁺ form). Elution is initially effected with water (500 ml) and subsequently with 5% ammonia solution (800 ml). The ammoniacal eluate is concentrated to dryness in vacuum. The residue is purified by means of column chromatography on silica gel, using the upper phase of an ethyl acetate/isopropanol/water (4/1/2, v/v/v) solvent mixture. The target compound, β-L-methyldeoxycytidine, is obtained as a yellowish foam (1.3 g), 200 mg of which is branched off, dissolved in a small amount of methanol and added with a few drops of methanolic HCl. The crystals having deposited after some time are filtrated off. The hydrochloride of β-L-methyldeoxycytidine obtained in this way has a melting point of 151-153° C. (dec.).

UV spectrum (H₂O, pH 7) λₘₐₓ 281 nm (ε 11644), λₘᵢₙ 251 nm (ε 5448); (H₂O, pH 1) λₘₐₓ 287 nm (ε 14770), λₘᵢₙ 245 nm (ε 2238).

¹H-NMR (Me₂SO-d₆) δ (ppm)=1.85 (s, 3H, CH₃), 1.92-1.97 (m, 1H, H-2′), 2.04-2.07 (m, 1H, H-2″), 3.42-3.57 (m, 2H, H5′ and H-5″), 3.74 (m, 1H, H-4′), 4.20 (m, 1H, H-3′), 5.07 (br d, 1H, OH-3′), 5.21 (br t, 1H, OH-5′), 6.15-6.17 (dd, 1H, H-1′), 6.77 (br s, 1H, NH), 7.26 (br s, 1H, NH), 7.61 (s, 1H, H-6).

¹³C-NMR (Me₂SO-d₆) δ (ppm)=13.3 (CH₃), 40.2 (C-2′), 61.4 (C-5′), 70.4 (C-3′), 84.6 (C-1′), 87.1 (C-4′), 101.27 (C-5), 138.30 (C-6), 155.20 (C-2), 165.31 (C-4).

Analysis calc. for C₁₀H₁₆N₃O₄Cl: C, 43.25; H, 5.81; N, 15.13.

Found: C, 43.11; H, 5.97; N, 14.89.

### 2. Synthesis of β-L-2′,3′-didehydro-2′,3′-dideoxy-5-methylcytidine (β-L-ddeMetC)

### 2.1 (2-Deoxy-β-L-ribofuranosyl)-N-benzoyl-5-methylcytosine

β-L-5-Methyldeoxycytidine (7.25 g, 30 mmol) under an argon protective atmosphere is dissolved in anhydrous dimethylformamide (200 ml) and added with benzoic anhydride (7.5 g, 33 mmol). The reaction mixture is stirred at room temperature for 24 hours. Subsequently, the solvent is removed in vacuum. The resulting residue is triturated with ether, filtrated off, washed with ether and dried. 9.3 g of 1-(2-deoxy-β-L-ribofuranosyl)-N-benzoyl-5-methylcytosine is obtained as a raw product.

### 2.2 1-(2-Deoxy-3,5-di-O-mesyl-β-L-ribofuranosyl)-N-benzoyl-5-methylcytosine

A solution of the above-described N-benzoyl derivative (2.2 g, 6.4 mmol) in pyridine (20 ml) is added with methanesulfonyl chloride (1.0 ml, 13.6 mmol) at −5° C. Following stirring for 18 hours, the solution is poured into an ice/water mixture (200 ml) with vigorous stirring. The precipitated product is air-dried. Yield: 2.9 g.

### 2.3 1-(2-Deoxy-3,5-epoxy-β-L-threo-pentofuranosyl)-5-methyl-cytosine

A solution of the di-O-mesyl compound described under 2.2 (2.26 g, 4.52 mmol) in ethanol (150 ml) and water (35 ml) containing sodium hydroxide (15 ml of a 1 N solution) is heated at reflux for two hours. Following cooling, the solution is neutralized with dilute acetic acid and concentrated to dryness. The residue is extracted several times with hot acetone (5×50 ml). The acetone solution is concentrated, and the resulting residue is suspended in cold ethanol (7 ml). The precipitated crystals are filtrated off (0.55 g).

### 2.4 1-(2,3-Dideoxy-β-L-glycero-pent-2-enofuranosyl)-5-methylcytosine (2′,3′-didehydro-2′,3′-dideoxy-5-metylcytidine; β-L-ddeMetC)

A solution of potassium tert-butylate (1.93 g, 17 mmol) in 55 ml of anhydrous dimethylsulfoxide is added with the 3′,5′-epoxy derivative of β-L-5-methylcytidine (1.75 g, 8.3 mmol) prepared under 2.3 and stirred for 3 hours at room temperature. The solvent is removed in vacuum, and the residue is dissolved in water (7.5 ml) and neutralized with acetic acid/methanol (1/1, v/v). The neutral solution is concentrated to dryness, and the residue is separated by means of column chromatography on silica gel, using chloroform/methanol (85/15, v/v) as eluent. Following stripping of the solvent, β-L-ddeMetC (0.72 g) is obtained from the corresponding fractions.

UV spectrum (H₂O, pH 7) λₘₐₓ 278 nm (ε 10067), λₘᵢₙ 253 (ε 5668); (H₂O, pH 1) λₘₐₓ 285 nm (ε 9240), λₘᵢₙ 244 nm (ε 1628).

¹H-NMR (Me₂SO-d₆) δ (ppm)=1.79 (s, 3H, CH₃), 3.57 (m, 2H, H-5′ and H-5″), 4.74 (m, 1H, H-4′), 4.97 (t, 1H, OH-5′), 5.86 (m, 1H, H-2′), 6.31 (m, 1H, H-3′), 6.79 (br s, 1H, NH) 6.89 (m, 1H, H-1′), 7.28 (br s, 1H, NH), 7.53 (s, 1H, H-6).

¹³C-NMR (Me₂SO-d₆) δ (ppm)=13.26 (CH₃), 62.59 (C-2′), 86.99 (C-5′), 89.56 (C-3′), 101.18 (C-1′), 126.92 (C-4′), 133.89 (C-5), 138.84 (C-6), 155.40 (C-2), 165.43 (C-4).

Analysis calc. for C₁₀H₁₃N₃O₃: C, 53.81; H, 5.87; N, 18.82. Found: C, 53.57; H, 5.76; N, 18.86.

### 3. Synthesis of 1-(2,3-dideoxy-β-L-glycero-pentofuranosyl)-5-methylcytosine (β-L-2′,3′-dideoxy-5-methylcytidine; β-L-ddMetC)

1-(2,3-Dideoxy-β-L-glycero-pent-2-enofuranosyl)-5-methylcytosine (0.22 g, 1 mmol) is dissolved in methanol (80 ml). Palladium/carbon (10%) is added, and hydrogenation is effected at room temperature. Monitoring by thin-layer chromatography shows absence of starting material after 30 minutes. The catalyst is filtrated off, the filtrate is concentrated, and the resulting residue is subjected to column chromatography on silica gel, using chloroform/methanol (9/1, v/v) as eluent. Corresponding fractions are combined. Following removal of the solvent, a residue is obtained which yields pure 1-(2,3-dideoxy-β-L-glycero-pentofuranosyl)-5-methylcytosine (L-ddMetC, 103 mg) after crystallization from methanol.

¹H-NMR (Me₂SO-d₆) δ (ppm)=1.67 (d, 3H, CH₃), 2.37-2.63 (m, 4H, H-2′, H-2″, H-3′ and H-3″), 3.78 (m, 2H, H-5′ and H-5″), 4.08 (m, 1H, H-4′), 5.10 (br t, 1H, 5′-OH), 6.85 (dd, 1H, H-1′), 7.12 (br s, 1H, NH), 7.23 (br s, 1H, NH), 7.64 (s, 1H, H-6).

¹³C-NMR (Me₂SO-d₆) δ (ppm) 13.7 (CH₃), 38.3 (C-2′), 65.4 (C-5′), 69.1 (C-3′), 81.3 (C-1′), 86.4 (C-4′), 116.2 (C-5), 137.7 (C-6), 153.5 (C-2), 164.7 (C-4).

Analysis calc. for C₁₀H₁₅N₃O₃: C, 53.31; H, 6.72; N, 18.66.

Found: C, 53.43; H, 6.70; N, 18.73.

### 4. Synthesis of 1-β-L-arabinofuranosyl-5-methylcytosine (β-L-AraMetC)

1-β-L-Ribofuranosylthymine is converted into the 5′-O-trityl ether with triphenylchloromethane in the usual manner. This compound (5.4 g, 10.8 mmol) is suspended in toluene (120 ml), heated to 80° C., and added with a solution of N,N′-thiocarbonyldiimidazole (2.16 g, 12.1 mmol) in toluene (80 ml). The mixture is heated at reflux for two hours. Following cooling, the solid material is filtrated off and recrystallized from ethanol. 2,2′-Anhydro-5′-O-trityl-L-ribo-furanosylthymine is obtained in an amount of 4.3 g. The anhydro compound (4.3 g, 10 mmol) is placed in 50% ethanol (400 ml), 1 N NaOH (25.5 ml) is added, and the suspension is heated to 60° C. for two hours. Once cooled down, the solution is neutralized with acetic acid. The ethanol is distilled off, and 1-(5′-O-trityl-β-L-arabinofuranosyl)thymine precipitates from the remaining aqueous portion. 4.4 g of this compound is obtained which is dissolved in methanol (250 ml) and added with concentrated hydrochloric acid (25 ml). Cleavage of the 5′-O-trityl group is complete after two hours. Following stripping of the solvent, the resulting residue is separated by means of column chromatography on silica gel, using a chloroform/methanol mixture (8/2, v/v) as eluent. 1-β-L-Arabinofuranosylthymine (1.7 g, 6,6 mmol) is obtained from the corresponding fractions. The conversion of this compound into 1-β-L-arabinofuranosyl-5-methylcytosine proceeds in an analogous manner as described under Example 1.1 and 1.2.

The melting point of the hydrochloride is 201-203° C. (dec.).

UV spectrum (H₂O, pH 7) λₘₐₓ 281 nm (ε 11796), λₘᵢₙ 250 nm (ε 4600); (H₂O, pH 1) λₘₐₓ 288 nm (ε 14633), λₘᵢₙ 247 nm (ε 3800).

¹H-NMR (Me₂SO-d₆) δ (ppm)=1.84 (s, 3H, CH₃), 3.60 (m, 2H, H-5′ and H-5″), 3.71 (d, 1H, H-4′), 3.88 (m, 1H, H-3′), 3.94 (m, 1H, H-2′), 5.05 (s, 1H, 5′-OH), 5.34 (s, 1H, 3′-OH), 5.38 (s, 1H, 2′-OH), 6.19 (d, 1H, H-1′), 6.71 (br s, 1H, NH), 7.20 (br s, 1H, NH), 7.42 (s, 1H, H-6).

¹³C-NMR (Me₂SO-d₆) δ (ppm)=13.3 (CH₃), 61.1 (C-5′), 74.9 (C-3′), 76.2 (C-2′), 84.6 (C-4′), 85.5 (C-1′), 99.2 (C-5), 140.2 (C-6), 155.2 (C-2), 165.2 (C-4).

Analysis calc. for C₁₀H₁₅N₃O₅: C, 46.69; H, 5.88; N, 16.33.

Found: C, 46.81; H, 5.64; N, 16.37.

### 5. Synthesis of 1-(3′-azido-2′,3′-dideoxy-L-ribofuranosyl)-5-methylcytosine) (β-L-3′-azido-2′,3′-dideoxy-5-methylcytidine; β-L-N₃MetCdR)

1-(3′-Azido-2′,3′-dideoxy-β-L-ribofuranosyl)thymine (3.1 g; 11.6 mmol) is dissolved in pyridine (50 ml), and acetic anhydride (2.8 ml; 30 mmol) is added with ice cooling. The mixture is maintained at room temperature for 12 hours with stirring. Subsequently, the solvent is removed in vacuum, and the residue is dissolved in chloroform. The solution is washed twice with saturated sodium hydrogen carbonate solution and with water. The organic phase is dried with sodium sulfate, the solvent is removed in vacuum, and the resulting residue is purified by means of column chromatography on silica gel, using chloroform/methanol (97/3, v/v) as eluent. The corresponding fractions yield 2.9 g of 1-(5′-O-acetyl-3′-azido-2′,3′-dideoxy-β-L-ribofuranosyl)thymine in the form of a foam (yield: 81%). This compound is dissolved in pyridine (50 ml) and added with 1H-1,2,4-triazole (1.37 g; 15 mmol) and chlorophenyl dichlorophosphate (2.42 ml; 15 mmol). The mixture is maintained at room temperature for four days with stirring. Thereafter, the pyridine is removed in vacuum, and the residue is dissolved in dioxane/25% ammonia (50 ml; 3/1, v/v) and stored at room temperature overnight. The solvent is removed in vacuum, and the resulting residue is dissolved in water (50 ml) and applied on a column (2×20 cm) containing DOWEX 50 WX 8 (H⁺ form). Elution is initially effected with water (600 ml) and subsequently with 5% aqueous ammonia solution (750 ml). The ammoniacal eluate is concentrated to dryness in vacuum, and the residue is purified by means of column chromatography on silica gel, using chloroform/methanol (9/1; v/v) as mobile phase. 1-(3′-Azido-2′,3′-dideoxy-β-L-ribofuranosyl)-5-methylcytidine is isolated from the corresponding fractions as a yellowish amorphous product (1.1 g; 41.7%).

¹H-NMR (Me₂SO-d₆) δ (ppm)=1.78 (s, 3H, CH₃), 1.98-2.09 (m, 2H, H-2′ and H-2″), 3.56-3.63 (m, 2H, H-5′ and H-5″), 3.86 (m, 1H, H-4′), 4.12 (m, 1H, H-3′), 5.17 (br t, 1H, 5′-OH), 6.15-6.18 (dd, 1H, H-1′), 6.83 (br s, 1H, NH), 7.56 (br s, 1H, NH), 7.86 (s, 1H, H-6).

¹³C-NMR (Me₂SO-d₆) δ (ppm)=20.2 (CH₃), 36.7 (C-2′), 60.8 (C-5′), 75.3 (C-3′), 84.7 (C-1′), 88.1 (C-4′), 108.2 (C-5), 140.5 (C-6), 152.7 (C-2), 164.3 (C-4).

### 6. Determination of the Antiviral Activity of β-L-5-methyl-cytosine nucleosides

The antiviral efficacy of the compounds of at least one embodiment was investigated on HepG2 2.2.15 cells, a human hepatoblastoma cell line which has the replication-competent HBV genome stably integrated therein and produces infectious progeny viruses in a productive manner (Sells et al., Proc Natl Acad Sci USA 1987, 84: 1005-1009).

The above cells were cultured under standardized conditions as specified by Korba and Gerin, and the amount of extracellular viral DNA was determined (Korba et al., Antiviral Res 1992, 19: 55-70).

Following passaging, the HepG2 2.2.15 cells were seeded at a density of about 60% in 12-well plates and cultured to confluence in 10% FBS Dulbecco MEM. Thereafter, the medium was changed to 2% FBS, and the cells were cultured for another 24 hours.

After another change of medium, the cells were treated with varying concentrations of compounds according to at least one embodiment. Every 24 hours the compounds were re-added together with the medium. On the 6th day of treatment, the cell supernatants were centrifuged off and stored at −20° C. until analysis of the HBV DNA was effected.

Following treatment of the culture supernatants with proteinase K, the extracellular viral DNA was amplified by means of PCR using the following primers (forward: 5′-CTC CAG TTC AGG AAC AGT AAA CCC-3′; reverse: 5′-TTG TGA GCT CAG AAA GGC CTT GTA AGT TGG CG-3′. The PCR products were separated on 1% agarose, stained with ethidium bromide and quantified using a Fluor-S™ Multimager (Biorad).

For calibration of the PCR reaction, serial dilutions of the pUC19 HBV and pTHBV plasmids with known genome equivalents (GE) were used, resulting in a lower detection limit of about 10³ GE and a linearity between 10³ and 10⁵ GE.

Table 1 shows the concentrations of compounds of at least one embodiment required for 50% reduction of extracellular HBV DNA (ED₅₀) after 9 days of incubation of the HepG2 2.2.15 cells.

TABLE 1
Efficacy of β-L-5-methylcytidine nucleosides on HBV
replication in HepG2 2.2.15 cells compared to 3TC
(lamivudine). The concentrations resulting in 50% reduction
of HBV DNA in the medium of the cells (ED₅₀ values) are given.
ED₅₀ values; μM

| 3TC | β-L- | β-L- | β-L- | β-L- | β-L |
|---|---|---|---|---|---|
| Lamivudine | MetCdR | ddMetC | AraMetC | N₃MetCdR | ddeMetC |
| 0.1-0.2 | 0.27-0.6 | >20 | >20 | 0.35-0.95 | >20 |

### 7. Inhibitability of HBV DNA polymerase by β-L-5-methylcytidine nucleoside triphosphates

Synthesis and purification of the triphosphates of β-L-5-methylcytidine nucleosides were effected according to well-known methods (Yoshikawa et al., Tetradedron Lett 1967, 50: 5065-5068; Hoard et Ott, J Am Chem Soc 1965, 87: 1785-1788).

To determine the endogenous HBV DNA polymerase activity, about 60 ml of serum from patients with hepatitis B virus infections from Charité, Berlin (>10⁷ HBV particles/ml), was centrifuged at 3000 rpm, followed by sedimentation of the virus particles at 25,000 g, 60 min, taking up in 7 ml of TKM buffer (50 mM Tris-HCl, pH 7.5, 50 mM KCl, 5 mM MgCl₂), and centrifugation through a saccharose gradient (0.3 M, 0.6 M, 0.9 M saccharose in 10 ml of TKM buffer each time) at 25,000 g for 20 hours. The purified virus sediment was suspended in 400 μl of TKM using ultrasound, divided in aliquots and frozen at −80° C. (Davies et al., Antiviral Res 1996, 30: 133-145).

30 μl of polymerase batch included about 2-4×10⁸ purified virus particles (lysed beforehand in 6% β-mercaptoethanol, 10% Igepal for 15 min at room temperature), 42 mM Tris-HCl, (pH 7.5), 34 mM MgCl₂, 340 mM KCl, 22 mM β-mercaptoethanol, 0.4% Igepal, 70 μM TTP, dATP, dGTP and 1 μCi ³H-dCTP (=0.7 μM dCTP) (Matthes et al., Antimicrob Agents & Chemoth 1991, 35: 1254-1257) and varying concentrations of β-L-5-methylcytidine nucleoside triphosphates.

Following a two-hour incubation at 37° C., 20 μl of each batch was placed on paper filter, washed 5 times with 5% trichloroacetic acid and 0.1% Na pyrophosphate, and the ³H-dCTP incorporated in the HBV DNA was subsequently measured in a Liquid Scintillation Counter.

Using the concentration-dependent inhibition curves of HBV DNA synthesis, the concentration of β-L-5-methylcytidine nucleoside triphosphates resulting in 50% inhibition of the HBV DNA polymerase activity was determined. The IC₅₀ values are illustrated in Table 2.

TABLE 2
Inhibition of HBV DNA polymerase by β-L-5-methylcytidine
nucleoside triphosphates compared to 3TC
triphosphate (IC₅₀ values).

| Triphosphate of | IC₅₀ [μM] |
|---|---|
| 3TC | 0.30 |
| (lamivudine) | |
| β-L-N₃MetCdR | 0.80 |
| β-L-MetCdR | 0.90 |
| β-L-ddeMetCdR | 6.2 |
| β-L-ddMetCdR | 6.5 |
| β-L-AraMetC | 10.8 |

### 8. Cytotoxicity of β-L-5-methylcytosine nucleosides

To this end, established cells of a human myeloid leukemia (HL-60) in RPMI medium, and the above-mentioned HepG2 cells in Dulbecco MEM, respectively, were incubated for two days using varying concentrations of compounds, and the proliferation rate of the cells was subsequently determined. The data was used to determine the concentration of compounds resulting in 50% inhibition of proliferation (CD₅₀).

TABLE 3
Cytotoxicity (CD₅₀) of β-L-5-methylcytidine
nucleosides to HL-60 and HepG2 cells.

| | | | | β-L |
|---|---|---|---|---|
| Cells: | β-L-MetCdR | β-L-N₃MetCdR | β-L-AraMetC | ddeMetCdR |
| HL-60 | >2000 | 2000 | >2000 | 600 |
| cells | | | | |
| Hep G2 | >2000 | >2000 | >2000 | 1500 |
| cells | | | | |

In the present application, if and when the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

The components disclosed in the various publications, disclosed or incorporated by reference herein, may possibly be used in possible embodiments of the present invention, as well as equivalents thereof.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

The appended drawings in their entirety, including all dimensions, proportions and/or shapes in at least one embodiment of the invention, are accurate and are hereby included by reference into this specification.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

All, or substantially all, of the components and methods of the various embodiments may be used with at least one embodiment or all of the embodiments, if more than one embodiment is described herein.

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

All of the patents, patent applications and publications recited herein, and in the Declaration attached hereto, are hereby incorporated by reference as if set forth in their entirety herein.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

It will be understood that the examples of patents, published patent applications, and other documents which are included in this application and which are referred to in paragraphs which state “Some examples of . . . which may possibly be used in at least one possible embodiment of the present application . . . ” may possibly not be used or useable in any one or more embodiments of the application.

The sentence immediately above relates to patents, published patent applications and other documents either incorporated by reference or not incorporated by reference.

All of the patents, patent applications or patent publications, which were cited in the International Search Report dated Feb. 14, 2005, and/or cited elsewhere are hereby incorporated by reference as if set forth in their entirety herein as follows: DE 195 18 216 A1, published Apr. 11, 1996; WO 94/27616 A, published Dec. 8, 1994; article by Van Draanen et al., published in 1994; article by Janta-Lipinski et al., published in 1998; and article by Cavalcanti et al., published in 1999.

The corresponding foreign and international patent publication applications, namely, Federal Republic of Germany Patent Applications No. 103 42 510.1 and No. 103 42 509.8, each filed on Sep. 12, 2003, and DE-OS 103 42 510.1, DE-OS 103 42 509.8, DE-PS 103 42 510.1, and DE-PS 103 42 509.8, and International Application No. PPCT/DE2004/002051, filed on Sep. 13, 2004, having WIPO Publication No. WO 2005/026186 and inventors Eckart MATTHES, Martin JANTA-LIPINSKI, Hans WILL, Hüseyin SIRMA, and Lin L I, are hereby incorporated by reference as if set forth in their entirety herein for the purpose of correcting and explaining any possible misinterpretations of the English translation thereof. In addition, the published equivalents of the above corresponding foreign and international patent publication applications, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The details in the patents, patent applications and publications may be considered to be incorporable, at applicant's option, into the claims during prosecution as further limitations in the claims to patentably distinguish any amended claims from any applied prior art.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The abstract of the disclosure is submitted herewith as required by 37 C.F.R. §1.72(b). As stated in 37 C.F.R. §1.72(b):
A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.

Therefore, any statements made relating to the abstract are not intended to limit the claims in any manner and should not be interpreted as limiting the claims in any manner.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

1-41. (canceled)

42. A β-L-5-methylcytosine nucleosides of formulas I or II for the treatment and prophylaxis of hepatitis B infections 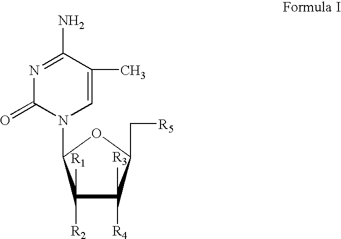
wherein
R₁═H, OH, or F
R₂═H or OH
R₃═H
R₄═H, OH, or N₃
and R₅═OH, O-acetyl, O-palmitoyl, alkoxycarbonyl, carbamate, phosphonate, monophosphate, diphosphate or triphosphate, and
if R₁, R₂ and R₃═H, then R₄═H, OH or N₃, and
if R₁═OH or F, then R₂ and R₃═H and R₄═OH, 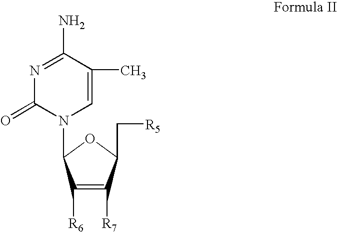
wherein
R₆═H, F, and R₇═H.

43. The β-L-nucleoside as claimed in claim 42, wherein the β-L-nucleoside is β-L-5-methyldeoxycytidine.

44. The β-L-nucleoside as claimed in claim 42, wherein the β-L-nucleoside is β-L-2,3′-dideoxy-5-methylcytidine.

45. The β-L-nucleoside as claimed in claim 42, wherein the β-L-nucleoside is β-L-arabinofuranosyl-5-methylcytosine.

46. The β-L-nucleoside as claimed in claim 42, wherein the β-L-nucleoside is β-L-2′-fluoroarabinofuranosyl-5-methylcytosine.

47. The β-L-nucleoside as claimed in claim 42, wherein the β-L-nucleoside is β-L-3′-azido-2′,3′-dideoxy-5-methylcytidine.

48. The β-L-nucleoside as claimed in claim 42, wherein the β-L-nucleoside is β-L-2′,3′-didehydro-2′,3′-dideoxy-5-methylcytidine.

49. The β-L-nucleoside as claimed in claim 42, wherein the β-L-nucleoside is β-L-2′,3′-didehydro-2′,3′-dideoxy-2′-fluoro-5-methylcytidine.

50. The β-L-nucleoside as claimed in claim 42, wherein the β-L-nucleoside is one of:
β-L-5-methyldeoxycytidine;
β-L-2,3′-dideoxy-5-methylcytidine;
β-L-arabinofuranosyl-5-methylcytosine;
β-L-2′-fluoroarabinofuranosyl-5-methylcytosine;
β-L-3′-azido-2′,3′-dideoxy-5-methylcytidine;
β-L-2′,3′-didehydro-2′,3′-dideoxy-5-methylcytidine; and
β-L-2′,3′-didehydro-2′,3′-dideoxy-2′-fluoro-5-methylcytidine.

51. The β-L-nucleoside according to claim 50, wherein the β-L-nucleoside is from the group comprising a salt, a phosphonate, a monophosphate, diphosphate, triphosphate, another ester or a salt of such ester.

52. Nucleic acids comprising as building block at least one β-L-nucleoside as claimed in claim 51.

53. A pharmaceutical agent comprising a β-L-nucleoside or a derivative and/or a nucleic acid, as claimed in claim 52, optionally together with conventional auxiliaries, preferably carriers, adjuvants and/or vehicles.

54. The pharmaceutical agent as claimed in claim 53, wherein at least one of (A), (B), (C), (D), (E), (F), (G), (H), and (I):
(A) the pharmaceutical agent further comprises one or more additional agents from the group of antiviral, fungicidal or antibacterial agents, anti-cancer agents and/or immunostimulators or immunomodulators;
(B) the antiviral agents are protease inhibitors and/or reverse transcriptase inhibitors and/or inhibitors of HBV-DNA polymerase, the immunostimulators bropirimine, anti-human alpha-interferon antibodies, IL-2, GM-CSF, interferons, diethyl dithiocarbamate, tumor necrosis factors, naltrexone, tuscarasol and/or rEPO;
(C) the pharmaceutical agent includes one or more additional anti-HBV-effective agents from the group comprising PMEA (adefovir-dipivoxil), famciclovir, penciclovir, diaminopurine-dioxolane (DAPD), clevudine (L-FMAU), entecavir, interferon or thymosin al and/or inhibitors of nucleocapsid formation, particularly heteroarylpyrimidines;
(D) the agents are pegylated;
(E) the pharmaceutical agent includes one or more additional agents capable of eliminating the function of cellular proteins essential to HBV growth; and
(F) the pharmaceutical agent is effective against hepatitis B viruses resistant to lamivudine or other cytosine nucleosides such as emtricitabine (L-FTC), L-ddC or L-ddeC.
(G) the pharmaceutical agent prevents cancer;
(H) the pharmaceutical agent prevents formation of liver carcinoma resulting from chronic hepatitis triggered by HBV; and
(I) the carriers are selected from the group comprising fillers, diluents, binders, humectants, disintegrants, dissolution retarders, absorption enhancers, wetting agents, adsorbents and/or lubricants.

55. Use of the β-L-nucleosides, the nucleic acid, and/or the pharmaceutical agent as claimed in claim 54 in the prophylaxis or therapy of a viral, bacterial, fungicidal and/or parasitic infection, or of cancer.

56. The use as claimed in claim 55, wherein at least one of (J), (K), (L), (M), (N), (O), (P), (Q), (R), (S), (T), (U), (V), (W), and (X):
(J) the viral disease is associated with hepatitis virus, HIV, bovine immunodeficiency virus, caprine arthritis-encephalitis virus, equine infectious anemia virus, ovine Maedi-Visna virus, Visna-Lenti virus, avian leukosis virus, human T cell leukemia virus, and/or feline immunodeficiency virus;
(K) the hepatitis virus is a hepatitis B or hepatitis D virus;
(L) the HIV is HIV-0, HIV-1 and/or HIV-2;
(M) the β-L-nucleoside, the nucleic acid, and/or the pharmaceutical agent are used as prodrug, as feed additive and/or drinking water additive;
(N) the agents are prepared and/or used in the form of a gel, poudrage, powder, tablet, sustained-release tablet, premix, emulsion, brew-up formulation, drops, concentrate, granulate, syrup, pellet, bolus, capsule, aerosol, spray and/or inhalant;
(O) the β-L-nucleoside, the nucleic acid, and/or the pharmaceutical agent are present in a preparation at a concentration of from 0.1 to 99.5, preferably from 0.5 to 95, more preferably from 20 to 80 wt.-%;
(P) the β-L-nucleoside, the nucleic acid, and/or the pharmaceutical agent are employed orally, rectally, subcutaneously, intravenously, intramuscularly, intraperitoneally and/or topically.
(Q) the β-L-nucleoside, the nucleic acid, and/or the pharmaceutical agent are used in overall amounts of from 0.05 to 500 mg/kg, preferably from 5 to 100 mg/kg body weight per 24 hours;
(R) the β-L-nucleoside and/or the nucleic acid are employed in a single administration of from 1 to 80, preferably from 3 to 30 mg/kg body weight;
(S) the β-L-nucleoside, the nucleic acid, and/or the pharmaceutical agent are distributed over 2 to 10, preferably 3 to 5 daily applications;
(T) 1 to 2 tablets are administered in each oral application;
(U) the β-L-nucleoside, the nucleic acid, and/or the pharmaceutical agent are used in combination with at least one other well-known pharmaceutical agent;
(V) the β-L-nucleoside, the nucleic acid, and/or the pharmaceutical agent enhance the therapeutic effect of said other pharmaceutical agents in a non-additive, additive or synergistic fashion, increase the therapeutic index and/or reduce the risk of toxicity inherent in the respective compound;
(W) the β-L-nucleoside, the nucleic acid, and/or the pharmaceutical agent are administered together with said other well-known pharmaceutical agents at a ratio of about 0.005 to 1; and
(X) at least one β-L-nucleoside is used in combination with 3-deazauridine.

57. Use of the β-L-nucleoside and/or the nucleic acid as claimed in claim 51 for the production of pharmaceutical agents.

58. A method for the treatment of a viral, bacterial, fungicidal and/or parasitic infection, or of cancer, wherein the β-L-nucleoside, the nucleic acid, and/or the pharmaceutical agent as claimed in claim 54 are contacted with an organism.

59. A kit comprising the β-L-nucleoside, the nucleic acid, and/or the pharmaceutical agent as claimed in claim 51, optionally together with information for combining the contents of the kit.

60. Use of the kit as claimed in claim 59 in the prophylaxis or therapy of viral diseases.

61. Use of the β-L-nucleoside as claimed in claim 51 as a drug.
